(19) United States
(12) Patent Application Publication
(10) Pub. No.: US 2019/0184034 A1
(43) Pub. Date: Jun. 20, 2019

(54) USE OF hM4Di IN THE TREATMENT OF SEIZURE DISORDERS

(71) Applicant: Ovid Therapeutics Inc., New York`, NY (US)

(72) Inventor: Matthew DURING, Weston, CT (US)

(21) Appl. No.: 16/227,326

(22) Filed: Dec. 20, 2018

Related U.S. Application Data
(60) Provisional application No. 62/608,207, filed on Dec. 20, 2017.
(60) Provisional application No. 62/635,871, filed on Feb. 27, 2018.

(51) Int. Cl. A61K 48/00; A61K 38/17; A61K 31/5513; A61K 31/55; A61P 25/08; A61M 37/00

(52) U.S. Cl. CPC A61K 48/0058; A61K 38/1787; A61K 31/5513; A61M 2037/0007; A61P 25/08; A61M 37/0092; C12N 2750/14143; A61K 31/55

(57) ABSTRACT

Methods and compositions for treating a seizure disorder are provided which include administering to the patient an adeno-associated virus vector encoding hM4Di for delivery of hM4Di to a target location, the vector including a human or murine CaMKII promoter, a woodchuck hepatitis virus post-transcriptional regulatory element, and a bovine growth hormone polyadenylation sequence, and administering to the patient a synthetic ligand which activates hM4Di. Administration of the vector and synthetic ligand to a patient with a seizure disorder is associated with reduced symptoms of the seizure disorder. In embodiments, ultrasound is applied to a target location in the patient's brain to enhance permeability of the patient's blood brain barrier to the vector and/or synthetic ligand.

### CROSS-REFERENCE TO RELATED APPLICATIONS

This application claims benefit of and priority to U.S. Provisional Application No. 62/608,207, filed Dec. 20, 2017 and U.S. Provisional Application No. 62/635,871, filed Feb. 27, 2018, which are incorporated by reference in their entireties.

### TECHNICAL FIELD

Treatment of seizure disorders using hM4Di, synthetic ligands and ultrasound.

### BACKGROUND

Seizure disorders typically involve abnormal nerve cell activity in the brain, causing seizures which may be manifested by periods of unusual behavior, sensations, convulsions, diminished consciousness and sometimes loss of consciousness. Seizures can be a symptom of many different disorders that can affect the brain. Epilepsy is a seizure disorder characterized by recurrent seizures. See, e.g., Blume et al., Epilepsia. 2001; 42:1212-1218. Epileptic seizures are usually marked by abnormal electrical discharges in the brain and typically manifested by sudden brief episodes of altered or diminished consciousness, involuntary movements, or convulsions.

Seizures can be categorized as focal seizures (also referred to as partial seizures) and generalized seizures. Focal seizures affect only one side of the brain, while generalized seizures affect both sides of the brain. Specific types of focal seizures include simple focal seizures, complex focal seizures, and secondarily generalized seizures. Simple focal seizures can be restricted or focused on a particular lobe (e.g., temporal lobe, frontal lobe, parietal lobe, or occipital lobe). Complex focal seizures generally affect a larger part of one hemisphere than simple focal seizures, but commonly originate in the temporal lobe or the frontal lobe. When a focal seizure spreads from one side (hemisphere) to both sides of the brain, the seizure is referred to as a secondarily generalized seizure. Specific types of generalized seizures include absences (also referred to as petit mal seizures), tonic seizures, atonic seizures, myoclonic seizures, tonic clonic seizures (also referred to as grand mal seizures), and clonic seizures.

Examples of seizure disorders include epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, focal cortical dysplasia, and increased seizure activity or breakthrough seizures (also called serial or cluster seizures). Seizure disorders can be associated with a sodium channel protein type 1 subunit alpha (Scn1a)-related disorder.

Brain tumors of all types can be associated with seizure disorders. Certain tumors are associated with a greater frequency of seizures. For example, gangliogliomas are slow growing benign tumors which may occur in the spinal cord and/or temporal lobes. Gangliogliomas are composed of both neoplastic glial and ganglion cells which are disorganized, variably cellular, and non-infiltrative. Gangliogliomas are commonly associated with seizures. Gliomas are brain tumors that develop from glial cells in the brain. Gliomas are classified into four grades (I, II, III and IV), and the treatment and prognosis depend upon the tumor grade. Low grade gliomas originate from two different types of brain cells: astrocytes and oligodendrocytes. Low grade gliomas are classified as a grade 2 tumor making them the slowest growing type of glioma. Between 60 and 85 percent of people with low-grade glioma may experience a seizure. High grade gliomas (grade 3 or 4) are fast growing gliomas that typically present a poor prognosis. Grade 3 gliomas include anaplastic astrocytoma, anaplastic oligodendroglioma, anaplastic oligoastrocytoma, and anaplastic ependymoma. Glioblastomas are grade 4 gliomas. Seizures occur in more than half of patients with grade III gliomas and about one-quarter of patients with grade IV gliomas. Meningiomas are tumors that arise from the meninges—the membranes surrounding the brain and spinal cord. Although not technically located in the brain, meningiomas may compress or squeeze the adjacent brain, nerves and vessels. Meningioma is the most common type of tumor that forms in the head. Most meningiomas are slow growing. Seizures are associated with meningiomas.

Focal cortical dysplasia is a malformation of cortical development, which is a common cause of medically refractory epilepsy in the pediatric population and a common etiology of medically intractable seizures in adults. Focal cortical dysplasia (FCD) has been classified into three types and further sub-types. Type I is typically associated with temporal lobes—malformation presenting with abnormal cortical lamination as a result of abnormal radial migration and maturation of neurons (FCD Type Ia) or disruption of typical 6-layered tangential composition of the cortex with immature neurons (FCD Type Ib) or both architectural abnormalities, radial and tangential cortical lamination (FCD Type Ic). Type II is commonly found in frontal lobes—malformation resulting from disrupted cortical lamination and specific cytological abnormalities, Type IIa—dysmorphic neurons (without balloon cells) and Type IIb—dysmorphic neurons and balloon cells. Type III—malformation connected with different cortical dislamination and cytological abnormalities with main lesion within the same area/lobe. Type IIIa—in the temporal lobe, cortical dislayering with hippocampal atrophy, IIIb—adjacent to glial or glioneuronal tumors (DNET, ganglioglioma), IIIc—adjacent to vascular malformations (as hemangiomas, arteriovenous malformations, telangiectasias, etc), IIId—acquired at early age (trauma, ischemia or perinatal hemorrhage, infectious or inflammatory diseases). See, Kabat and Krol, Pol J Radiol, 2012, 77(2) 35-43. FCD may involve any part of the brain, may vary in size and location and may be multifocal. Seizures are the main symptom of FCD, sometimes associated with mental retardation, particularly with early seizure onset. Symptoms can appear at any age, mostly in childhood, but also can occur in adults. Seizures associated with FCD can be drug-resistant.

Hemartomas are a mostly benign, focal malformation that resembles a neoplasm in the tissue of its origin. They are composed of tissue elements normally found at that site, but grow in a disorganized manner. Hemartomas can originate in the brain. Tuberous Sclerosis Complex (TSC) is a genetic seizure disorder characterized by hamartomatous growth in various organs. Patients who have this disorder can exhibit a high rate of epilepsy and cognitive problems resulting from multiple lesions in the brain. TSC lesions (corticol tubers) typically contain dysmorphic neurons, brightly eosinophilic giant cells and white matter alterations. Seizures associated with TSC can be intractable. Tuber cinereum hamartoma (also known as hypothalamic hamartoma) is a benign tumor in which a disorganized collection of neurons and glia accumulate at the tuber cinereum of the hypothalamus. Symptoms include gelastic seizures, a disorder characterized by spells of involuntary laughter with interval irritability and depressed mood.

Medications used to treat seizure disorders can be referred to as anti-epileptic drugs (“AED”). The treatment of recurrent seizures predominantly centers on the utilization of at least one AED, with possible adjunctive use of a second or even third agent in the case of monotherapeutic failure. See, Tolman and Faulkner, Ther Clin Risk Manag. 2011; 7: 367-375. However, approximately 30%-40% of epileptic patients have inadequate seizure control with just one AED, and require the use of adjunctive agents. Id. A subset of this group will have regular and persistent seizure activity despite reasonable doses of multiple AEDs. These seizures are considered refractory to treatment. Id. Accordingly, there remains a need for improved and/or additional therapies for treating seizure disorders.

Designer Receptors Exclusively Activated by Designer Drugs (DREADDS), otherwise known as RASSLs (receptor activated solely by synthetic ligands) have recently been proposed for treatment of seizures. See, e.g., US Publication Number US2016/0375097. DREADDS can involve engineered G protein coupled signal receptors (GPCRs) which are activated solely by synthetic ligands. These engineered receptors do not respond to endogenous ligands, but can still be activated by specific non-naturally occurring small-molecule drugs. Two types of DREADDS have been derived from human muscarinic acetylcholine receptors: hM3Dq which activates neuronal firing, and hM4Di (also referred to as hM4D(Gi)) which inhibits neuronal firing. Typically, native human muscarinic acetylcholine receptors are activated by the native ligand, acetylcholine. hM3Dq and hM4Di are not activated by the native ligand, but are activated by clozapine N-oxide (CNO), a pharmacologically inert ligand.

The blood brain barrier (BBB) prevents many compounds in the blood stream from entering the tissues and fluids of the brain. The BBB is formed by brain-specific endothelial cells and supported by the cells of the neurovascular unit to limit the passage of polar molecules or large molecules such as proteins and peptides into or out of the brain interstitium. However, the BBB also prevents many therapeutic compounds from entering the brain which can interfere with effective treatment of brain conditions and diseases. The BBB may interfere with delivery of DREADDS and/or ligands to the brain and thus reduce or prevent a therapeutic benefit in vivo.

One method of assisting transport of therapeutic drugs through the BBB involves delivering ultrasound energy to the BBB which “opens up” the BBB and interferes with the ability of the BBB to prevent transport of therapeutic agents into the brain. See, e.g., U.S. Pat. No. 5,752,515, which is directed to image guided ultrasound delivery of compounds through the BBB. In one aspect, the change induced in the central nervous system (CNS) tissues and/or fluids by ultrasound is by heating or cavitation. Such heating or cavitation may present a drawback since it may cause damage to tissues and potentially degrade the compounds being delivered for therapeutic benefit. Ultrasound also causes degradation of organic compounds. See, e.g., Bremner et al., Current Organic Chemistry, 15(2): 168-177 (2011) (“Bremner et al.”). According to Bremner et al., when aqueous solutions are irradiated with ultrasound, the H—O bond in water is homolytically cleaved to form hydroxyl radicals and hydrogen atoms. This process is the result of cavitation, whereby very high temperatures and pressures are generated within an imploding bubble. Id. Accordingly, use of ultrasound in an attempt to open the BBB to cause or increase delivery of therapeutic compounds to the brain such as the DREADDS and ligands discussed above could degrade them and interfere with or prevent therapeutic treatment.

### SUMMARY

A method of treating a seizure disorder in a patient in need thereof is provided which includes administering to the patient a vector which encodes a modified receptor for delivery of the modified receptor to a target location, the modified receptor being modified to be activated by a synthetic ligand, wherein the modified receptor inhibits neuronal firing when activated, and administering to the patient the synthetic ligand. In embodiments, the modified receptor is hM4Di. In embodiments, the vector includes a promoter such as a CaMKII (also referred to as murine CaMKII) promoter. In embodiments, the vector includes a promoter such as a human CaMKII (also referred to herein as human CaMK2a or hCaMK2a) promoter. In embodiments, the vector includes a post-transcriptional regulatory element such as Woodchuck Hepatitis Virus Post-Transcriptional Regulatory Element (WPRE). In embodiments, the vector includes a polyadenylation sequence such as bovine growth hormone polyadenylation sequence (BGHpA). In embodiments, the vector includes inverted terminal repeats (ITRs). In embodiments, the vector includes an origin of replication such as SV40/Ori. In embodiments, the vector includes an origin of replication such as pUC19/Ori. In embodiments, the vector includes an ampicillin resistance gene (Amp^{R}). In embodiments, the vector includes a fluorescence reporter gene. In embodiments, the vector is an adeno-associated virus. In embodiments, the vector is a lentivirus. In embodiments, the vector is pAM/CaMKII-hM4D(Gi)-WPRE-BGHpA. In embodiments, the vector is pAM/hCaMK2A-hM4D(Gi)-WPRE-BGHpA. In embodiments, the synthetic ligand is clozapine N-oxide. In embodiments, the synthetic ligand is perlapine. In embodiments, the synthetic ligand is administered orally, buccally, sublingually, rectally, topically, intranasally, vaginally or parenterally.

In embodiments, the vector is delivered to a target location in the patient's brain. In embodiments, the target location is the frontal lobe, the temporal lobe, the occipital lobe or the parietal lobe. In embodiments, the route of administration of the vector is oral, buccal, sublingual, rectal, topical, intranasal, vaginal or parenteral. In embodiments, the vector is administered directly to the target location through a patient's skull.

In embodiments, ultrasound is applied to a target location in the patient's brain to enhance permeability of the patient's blood brain barrier at the target location. A method of treating a seizure disorder in a patient in need thereof is provided which includes applying ultrasound to a target location in the patient's brain to enhance permeability of the patient's blood brain barrier at the target location; administering to the patient a vector encoding a modified receptor for delivery of the modified receptor to the target location, the modified receptor being modified to be activated by a synthetic ligand, wherein the modified receptor inhibits neuronal firing when activated; and administering to the patient the synthetic ligand. In embodiments, ultrasound is administered prior to administering the vector to the patient. In embodiments, the vector is administered to the patient prior to administering ultrasound to the patient. In embodiments, ultrasound is administered to the patient prior to administering the synthetic ligand. In embodiments, the method includes introducing a contrast agent into the patient, allowing sufficient time for the contrast agent to permeate the blood brain barrier and determining whether the contrast agent is present in the target location.

In embodiments, administration of the vector and synthetic ligand to a patient with a seizure disorder is associated with reduced symptoms of the seizure disorder. In embodiments, the seizure disorder is characterized by focal seizures. In embodiments, the seizure disorder is focal cortical dysplasia. In embodiments, the seizure disorder is epilepsy, epilepsy with generalized tonic-clonic seizures, epilepsy with myoclonic absences, frontal lobe epilepsy, temporal lobe epilepsy, occipital lobe epilepsy, parietal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, brain tumor induced seizures, hamartoma induced seizures, drug withdrawal induced seizures, alcohol withdrawal induced seizures, increased seizure activity or breakthrough seizures. In embodiments, the seizure disorder is associated with a sodium channel protein type 1 subunit alpha (Scn1a)-related disorder.

### BRIEF DESCRIPTION OF THE DRAWINGS

FIG. 1 is a plasmid map of pAM/CaMKII-hM4D(Gi)-WPRE-BGHpA.

FIGS. 2A, 2B, 2C and 2D depict the nucleotide sequence of pAM/CaMKII-hM4D(Gi)-WPRE-BGHpA [SEQ ID NO:1].

FIG. 3 depicts the amino acid sequence of AAVRec3 [SEQ ID NO:2].

FIG. 4 is a plasmid map of pAM/hCaMK2A-hM4D(Gi)-WPRE-BGHpA.

FIGS. 5A, 5B, 5C and 5D depict the nucleotide sequence of pAM/hCaMK2A-hM4D(Gi)-WPRE-BGHpA [SEQ ID NO:3].

FIGS. 6A, 6B, 6C, 6D, 6E, 6F, 6G, 6H, 6I, 6J, 6K, 6L and 6M are a schematic depiction showing correspondence between the location of components of pAM/hCaMK2A-hM4D(Gi)-WPRE-BGHpA and the nucleotide sequence [SEQ ID NO:3].

### DETAILED DESCRIPTION

Described herein are methods and compositions for treating a seizure disorder which include administering to the patient a vector encoding a modified receptor for delivery of the modified receptor to a target location, the modified receptor being modified to be activated by a synthetic ligand, wherein the modified receptor inhibits neuronal firing when activated; and administering to the patient the synthetic ligand. In embodiments, vectors encoding hM4Di are administered to a patient having a seizure disorder. In embodiments, the modified receptor is hM4Di. In embodiments, after vectors encoding hM4Di are delivered to a target location in a patient's brain, a synthetic ligand to hM4Di is administered to the patient and the patient exhibits improvement in one or more symptoms of the seizure disorder. In embodiments, ultrasound is applied to a target location in the patient's brain to enhance permeability of the patient's blood brain barrier at a target location, wherein hM4Di and/or the synthetic ligand are delivered to the target location.

The modified receptor hM4Di is derived from naturally occurring muscarinic receptors normally activated by the endogenous ligand, acetylcholine. The naturally occurring receptor is modified so as not to be activated by the endogenous ligand but is activated instead by a synthetic ligand, clozapine-N-oxide (CNO) or perlapine. The DREADD allows non-invasive control of neuron signaling through the Gᵢ (hM4Di) G-protein coupled signaling pathway. hM4Di is derived from the human muscarininc acetylcholine M4 receptor and inhibits neuronal firing. Without wishing to be bound by any theory, it is believed that hM4Di inhibits neuronal firing upon CNO or perlapine stimulation via activation of G-protein inwardly rectifying potassium (GIRK) channels. When bound by CNO or perlapine, membrane hyperpolarization results through a decrease in cAMP signaling and increased activation of inward rectifying potassium channels. This yields a temporary suppression of neuronal activity. A plasmid encoding hM4Di (pcDNA5/FRT-HA-hM4D(Gi)) is commercially available from Addgene, 75 Sidney Street, Cambridge, Mass. 02139 as plasmid 45548.

Nucleic acid constructs for expression of hM4Di may be produced recombinantly. Such expression vectors are provided herein. Expression vectors are a carrier nucleic acid into which a nucleic acid sequence can be inserted for introduction into a cell where it can be replicated. Expression vectors include plasmids, cosmids, recombinant viruses, such as adeno-associated virus (AAV), adenoviruses, retroviruses, poxviruses, and other known viruses in the art (bacteriophage, animal viruses, and plant viruses), and artificial chromosomes (e.g., YACs). A person of ordinary skill in the art is well equipped to construct expression vectors through standard recombinant techniques. In embodiments, an expression vector including nucleic acid encoding hM4Di is delivered to cells of a patient. The nucleic acid molecules are delivered to the cells of a patient in a form in which they can be taken up and are advantageously expressed so that therapeutically effective levels can be achieved. Upon such delivery, neurons in the target locations are transfected with hM4Di and can be made to respond to synthetic ligands so that therapeutically effective levels of inhibition can be achieved.

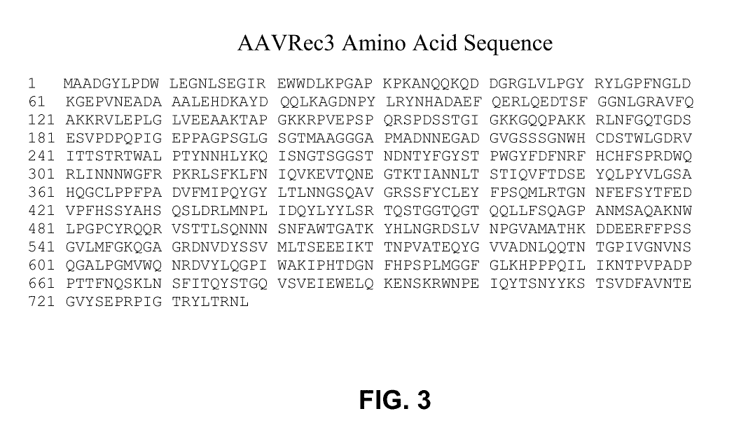

In embodiments, AAV vectors may be utilized herein to deliver hM4Di to the brain. AAV is a defective parvovirus known to infect many cell types and is nonpathogenic to humans. AAV can infect both dividing and non-dividing cells. Any of the known adeno-associated viruses (AAV) may be utilized herein, e.g., AAV1, AAV2, AAV4, AAV5, AAV8, AAV9 and AAVRec3 may be utilized in connection with neurons. Additional suitable AAV serotypes have been developed through pseudotyping, i.e., mixing the capsid and genome from different viral serotypes. Accordingly, e.g., AAV2/7 indicates a virus containing the genome of serotype 2 packaged in the capsid from serotype 7. Other examples are AAV2/5, AAV2/8, AAV2/9, etc. Hybrid AAV capsid serotypes rec1, rec2, rec3 and rec4 were generated by shuffling the fragments of capsid sequences that matched in all three non-human primate AAV serotypes cy5, rh20 and rh39, with AAV8. See, Charbel et al., PLoS One. 2013 Apr. 9; 8(4):e60361. The terms rec3AAV and AAVRec3 may be used interchangeably herein. The amino acid sequence of AAVRec3 is depicted in FIG. 3 [SEQ ID NO: 2]. Self-complementary adeno-associated virus (scAAV) may also be utilized as vectors. Whereas AAV packages a single strand of DNA and requires the process of second-strand synthesis, scAAV′ packages both strands which anneal together to form double stranded DNA. By skipping second strand synthesis scAAV allows for rapid expression in the cell.

Suitable vectors may be constructed by those having ordinary skill in the art using known techniques. Suitable vectors can be chosen or constructed, containing, in addition to hM4Di, appropriate regulatory sequences, including promoter sequences, terminator fragments, polyadenylation sequences, marker genes and other sequences as appropriate. Those skilled in the art are familiar with appropriate regulatory sequences, including promoter sequences, terminator fragments, polyadenylation sequences, marker genes and other suitable sequences.

Expression vectors herein include appropriate sequences operably linked to the coding sequence or ORF to promote its expression in a targeted host cell. “Operably linked” sequences include both expression control sequences such as promoters that are contiguous with the coding sequences and expression control sequences that act in trans or distally to control the expression of the desired product.

Typically, the vector includes a promoter to facilitate expression of hM4Di within a target cell. The promoter may be selected from a number of constitutive or inducible promoters that can drive expression of the selected transgene in the brain. Examples of constitutive promoters include a murine or human CaMKII promoter, CMV immediate early enhancer/chicken beta-actin (CBA) promoter-exon 1-intron 1 element, RSV LTR promoter/enhancer, the SV40 promoter, the CMV promoter, dihydrofolate reductase (DHFR) promoter, and the phosphoglycerol kinase (PGK) promoter.

Specificity can be achieved by regional and cell-type specific expression of the receptor exclusively, e.g., using a tissue or region specific promoter. Virus gene promoter elements may help dictate the type of cells that express hM4Di. Some promoters are nonspecific (e.g., CAG, a synthetic promoter), while others are neuronal-specific. The CAG promoter is a strong synthetic promoter that can be used to drive high levels of expression. The CAG promoter consists of 1) a cytomegalovirus (CMV) early enhancer element, 2) the promoter, the first exon and the first intron of the chicken beta-actin gene, and 3) the splice acceptor of the rabbit beta-globin gene. In embodiments the promoter is the CAG promoter. Neuronal specific promoters (e.g., synapsin; hSyn), are preferential to specific neuron types, e.g., dynorphin, encephalin, GFAP (Glial fibrillary acidic protein) which is preferential to astrocytes, or CaMKII, which is preferential to cortical glutamatergic cells but can also target subcortical GABAergic cells. In embodiments, the promoter is the murine or human CamkII (alpha CaM kinase II gene) promoter, which may drive expression in the forebrain. In embodiments, the CaMKII promoter is derived from murine α-Calcium/calmodulin-dependent kinase II (CaMKII), a gene with expression directed to excitatory neurons in the neocortex and hippocampus. As used herein, unless otherwise stated, “CaMKII” refers to murine CaMKII. In embodiments, the CaMKII promoter is derived from human α-Calcium/calmodulin-dependent kinase II (hCaMKII). The hCaMKII promoter is also referred to herein as the “human CaMK2A promoter” or the “hCaMK2A promoter”. Other neuronal cell type-specific promoters include the NSE promoter, tyrosine hydroxylase promoter, myelin basic protein promoter, glial fibrillary acidic protein promoter, and neurofilaments gene (heavy, medium, light) promoters.

Expression control sequences may also include appropriate transcription initiation, termination, and enhancer sequences; efficient processing signals such as splicing and polyadenylation signals; sequences that stabilize cytoplasmic nucleic acids; sequences that enhance translation efficiency (e.g., Kozak consensus sequence); sequences that enhance nucleic acid or protein stability; and when desired, sequences that enhance product processing and/or secretion. Many varied expression control sequences, including native and non-native, constitutive, inducible and/or tissue-specific, are known in the art and may be utilized herein depending upon the type of expression desired.

In addition to promoters, expression control sequences for eukaryotic cells typically include an enhancer, such as one derived from an immunoglobulin gene, SV40, CMV, etc., and a polyadenylation sequence which may include splice donor and acceptor sites. The polyadenylation sequence generally is inserted 3′ to the coding sequence and 5′ to the 3′ ITR sequence. Illustrative examples of polyA signals that can be used in a vector herein include polyA sequence (e.g., AATAAA, ATTAAA, or AGTAAA), a bovine growth hormone polyA sequence (BGHpA), a rabbit beta-globin polyA sequence (rBgpA), or another suitable heterologous or endogenous polyA sequence known in the art.

Regulatory sequences useful herein may also contain an intron, such as one located between the promoter/enhancer sequence and the coding sequence. One useful intron sequence is derived from SV40, and is referred to as the SV40 T intron sequence. Another includes the woodchuck hepatitis virus post-transcriptional element (WPRE). WPRE is a DNA sequence that, when transcribed, creates a tertiary structure that enhances expression.

Vectors herein may contain reporter genes, e.g., those which encode fluorophores. A fluorophore is a fluorescent compound that can re-emit light upon excitation, usually at specific frequencies. They can be used as a tag or marker which can be attached to, e.g., a protein to allow the protein to be located. Many suitable fluorophores are known in the art. They may be categorized by the color they emit, e.g., blue, cyan, green, yellow, orange, red and others. For example, mCherry, mRasberry, mTomato and mRuby are red fluorophore proteins; citrine, venus, and EYFP are yellow fluorophore proteins. Green fluorescent protein (GFP) is a commonly used fluorophore.

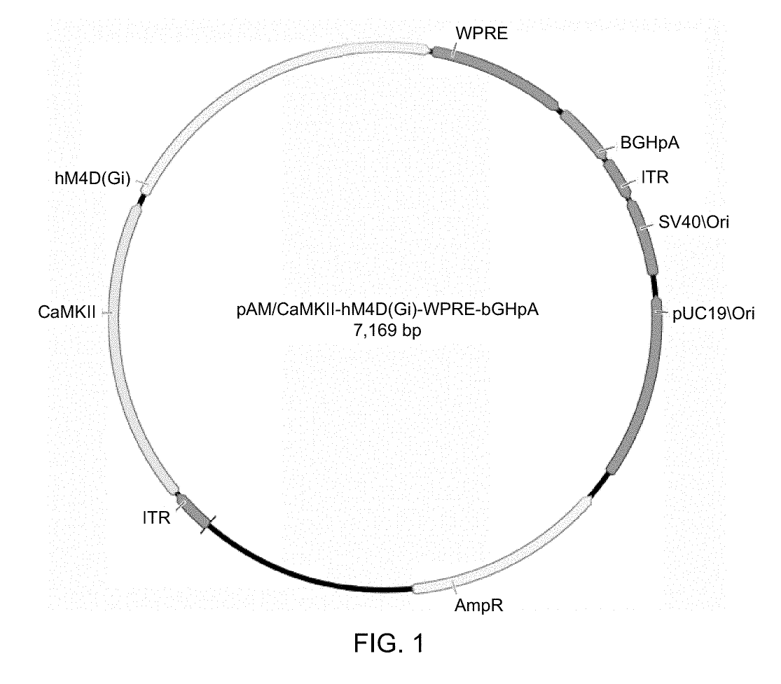

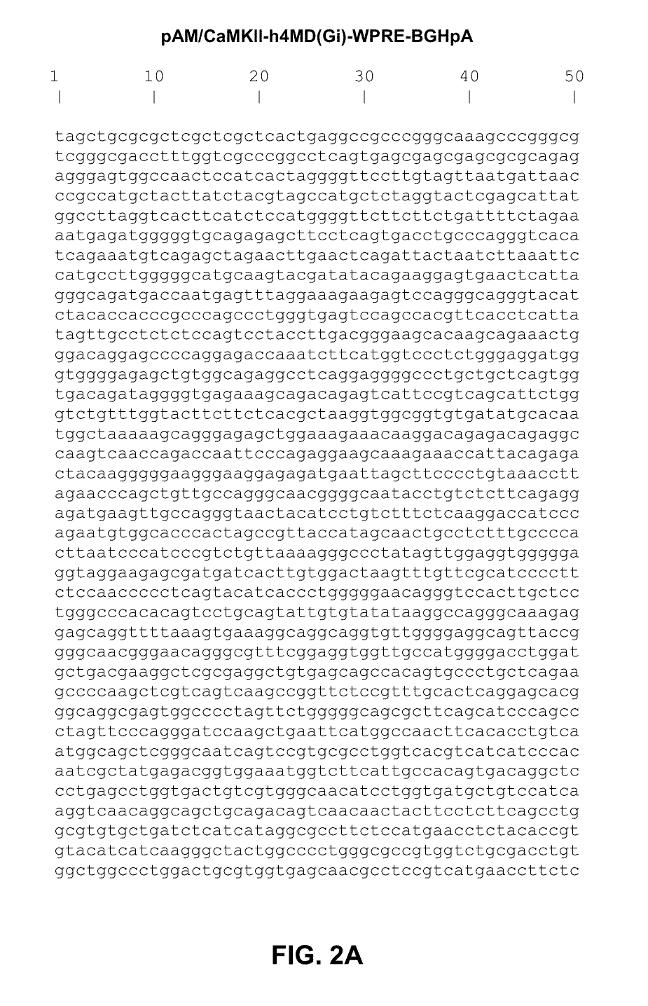

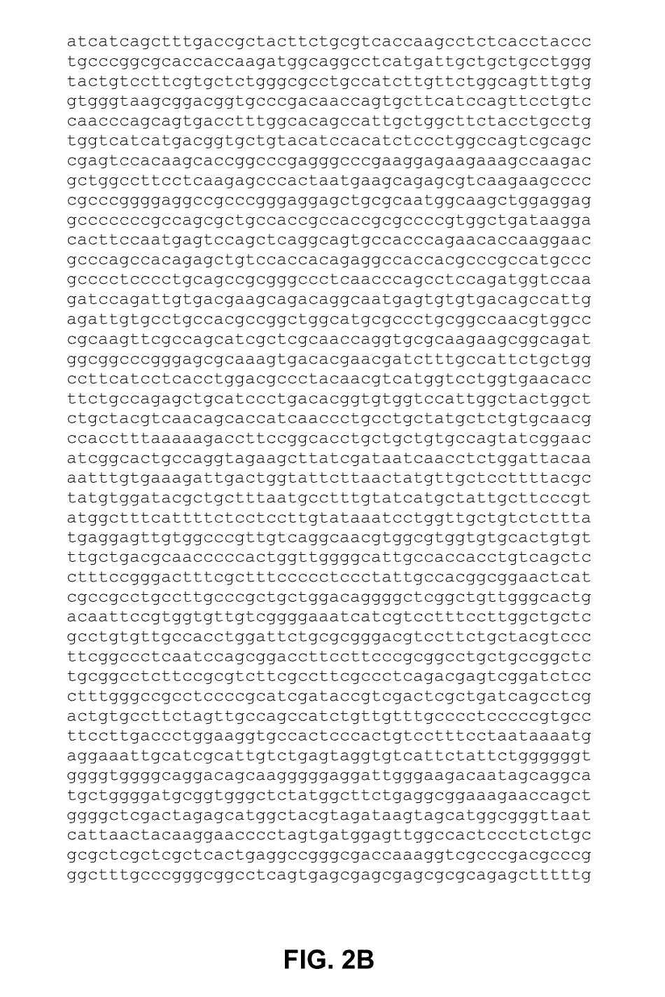

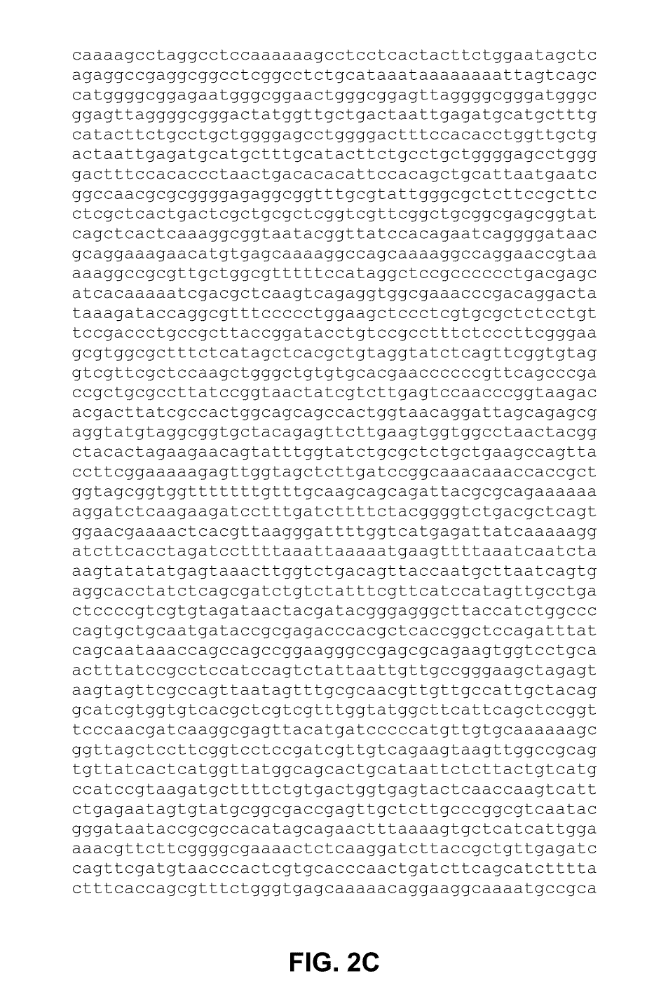

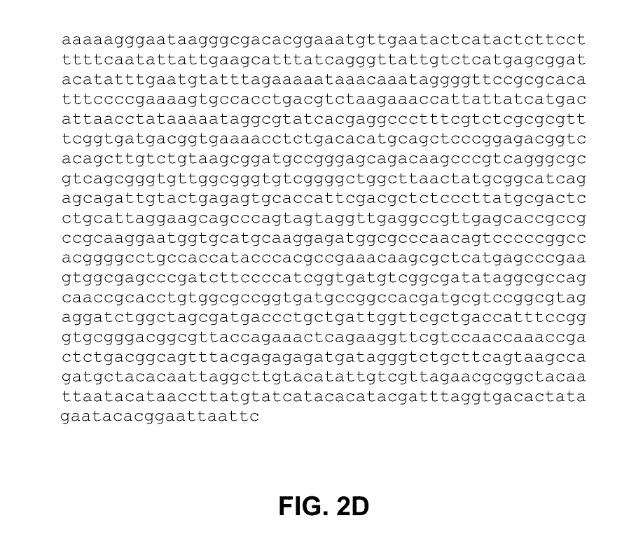

In embodiments, the expression vector is pAM/CaMKII-hM4D(Gi)-WPRE-BGHpA. A plasmid map of pAM/CaMKII-hM4D(Gi)-WPRE-BGHpA is depicted in FIG. 1. The nucleic acid sequence [SEQ ID NO:1] is shown in FIGS. 2A, 2B, 2C and 2D. TABLE I annotates pAM/CaMKII-hM4D(Gi)-WPRE-BGHpA.

TABLE I

| | | | | | Direc- |
|---|---|---|---|---|---|
| Name | Type | Minimum | Maximum | Length | tion |
| AmpR | CDS | 5381 | 6241 | 861 | reverse |
| pUC19\Ori | rep_origin | 4468 | 5255 | 788 | reverse |
| SV40\Ori | rep_origin | 4047 | 4382 | 336 | reverse |
| ITR | LTR | 3864 | 4046 | 183 | forward |
| BGHpA | polyA_signal | 3586 | 3854 | 269 | forward |
| WPRE | misc_feature | 2975 | 3567 | 593 | forward |
| hM4D(Gi) | CDS | 1530 | 2968 | 1439 | forward |
| CaMKII | promoter | 195 | 1483 | 1289 | forward |
| ITR | LTR | 1 | 183 | 183 | forward |

To construct pAM/CaMKII-hM4D(Gi)-WPRE-BGHpA, a fragment containing full length hM4D(Gi) coding region (1,437 bp) from the pAAV-CaMKIIa-hM4D(Gi)-mCherry plasmid (Cat. No:50477, Depositing lab Bryan Roth) commercially available from AddGene, 75 Sidney Street, Cambridge, Mass. 02139, is amplified by PCR using the following primers:

Forward
[SEQ ID NO: 4]
ATC TAG GAA TTC ATG GCC AAC TTC ACA CCT GTC
Reverse
[SEQ ID NO: 5]
ATC TAG AAG CTT CT A CCT GGC AGT GCC GAT GTT CCG

The Forward primer is extended with EcoRI restriction site and with 6 protective nucleotides. The Reverse primer is extended with the hM4D original TAG stop codon (reverse complement—CTA) followed by HindIII restriction site and 6 protective nucleotides. 1,461 bp PCR fragment is cut with EcoRI+HindIII and inserted into plasmid pAM/CaMKII-pL-WPRE-bGHpA cut with the same restriction enzymes. Integrity of the expression cassette is confirmed by sequencing.

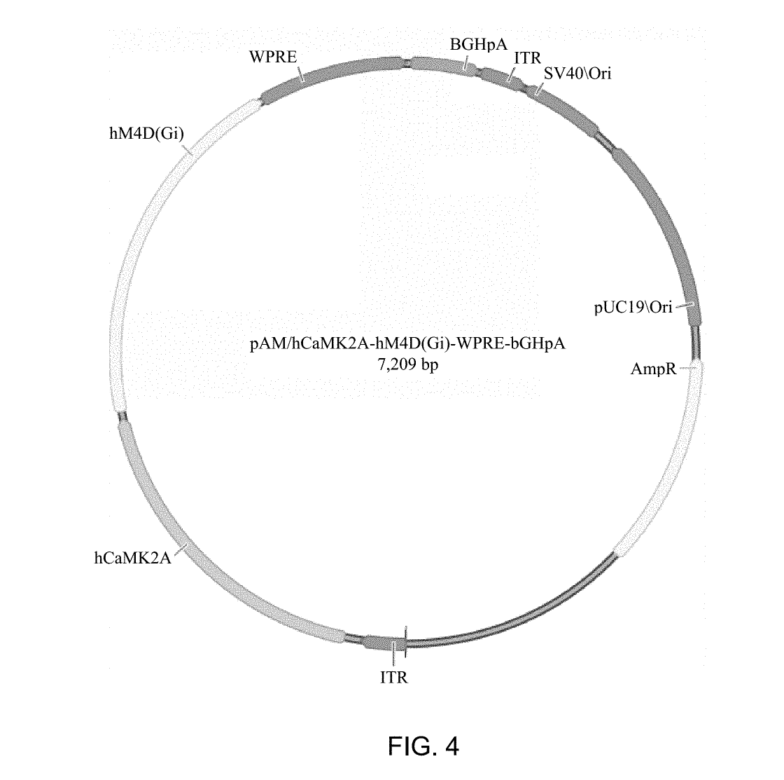

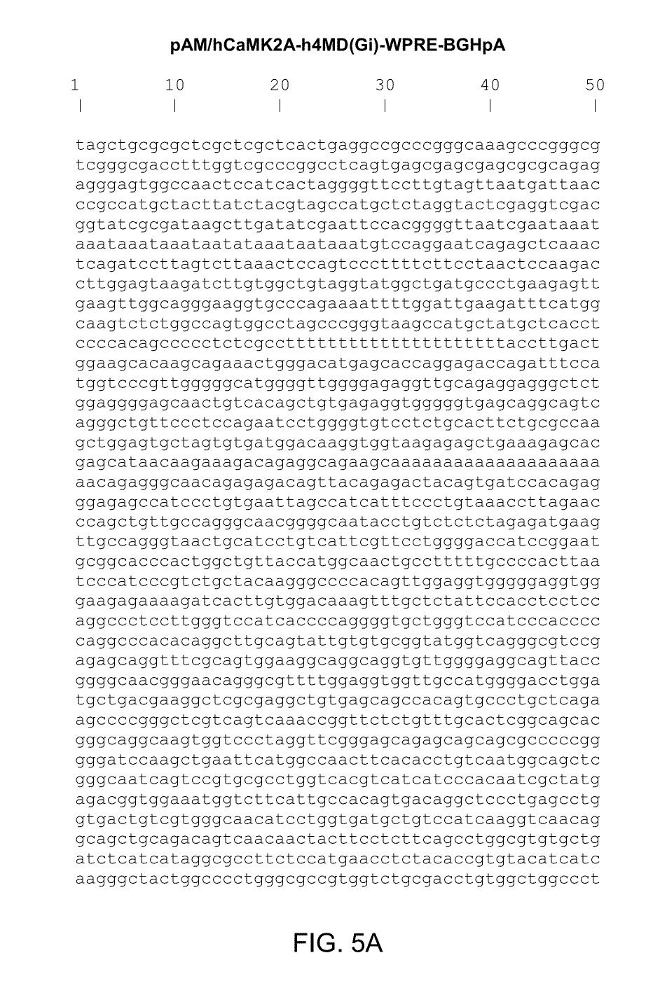

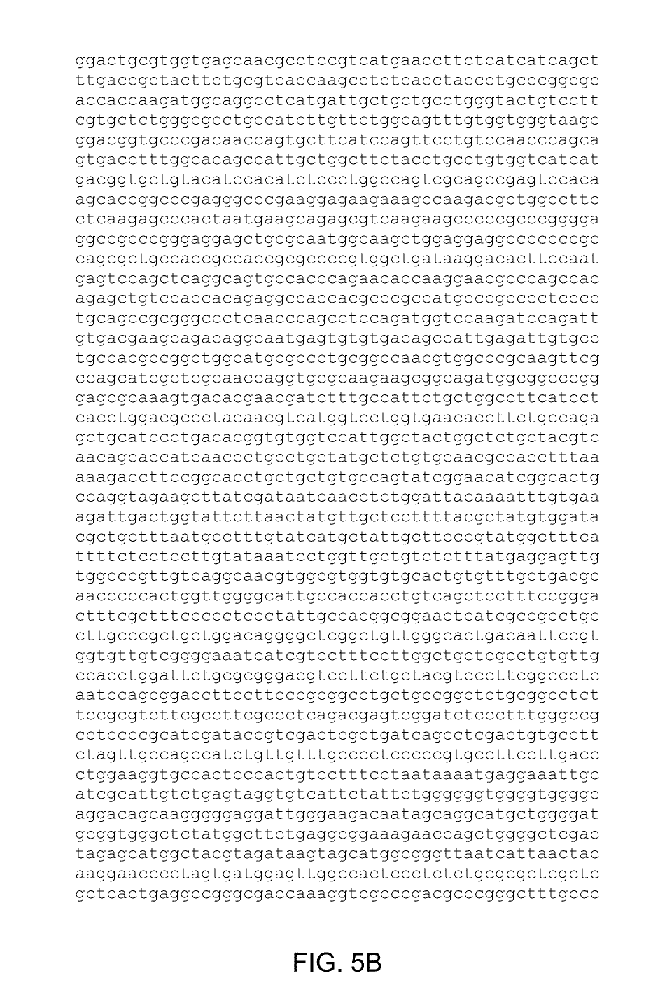

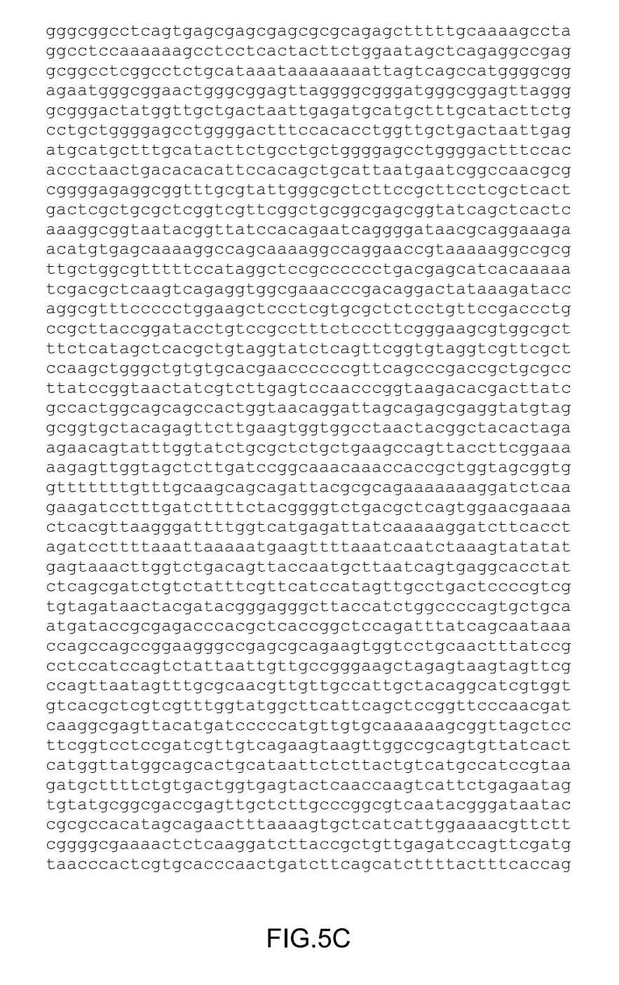

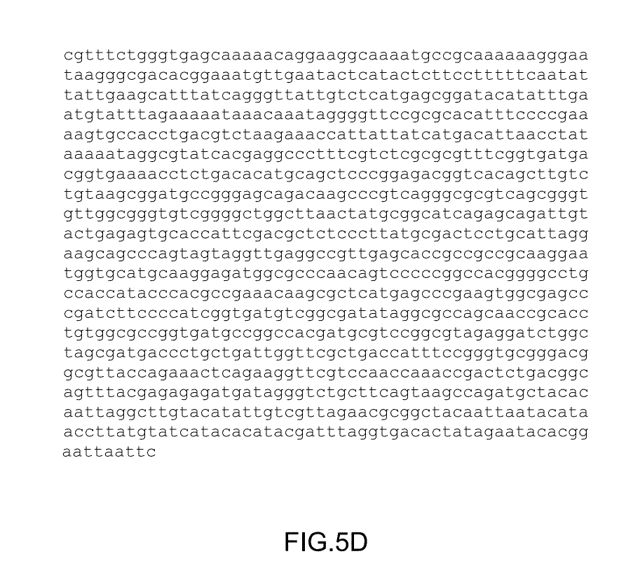

In embodiments, the expression vector is pAM/hCaMK2A-hM4D(Gi)-WPRE-BGHpA. A plasmid map of pAM/hCaMK2A-hM4D(Gi)-WPRE-BGHpA is depicted in FIG. 4. The nucleic acid sequence [SEQ ID NO:3] is shown in FIGS. 5A, 5B and 5C. TABLE II annotates pAM/hCaMK2A-hM4D(Gi)-WPRE-BGHpA.

TABLE II

| | | | | | Direc- |
|---|---|---|---|---|---|
| Name | Type | Minimum | Maximum | Length | tion |
| AmpR | CDS | 5421 | 6281 | 861 | reverse |
| pUC19\Ori | rep_origin | 4508 | 5295 | 788 | reverse |
| SV40\Ori | rep_origin | 4087 | 4422 | 336 | reverse |
| ITR | LTR | 3904 | 4086 | 183 | forward |
| BGHpA | polyA_signal | 3626 | 3894 | 269 | forward |
| WPRE | misc_feature | 3015 | 3607 | 593 | forward |
| hM4D(Gi) | CDS | 1570 | 3008 | 1439 | forward |
| hCaMK2A | promoter | 244 | 1545 | 1302 | forward |
| ITR | LTR | 1 | 183 | 183 | forward |

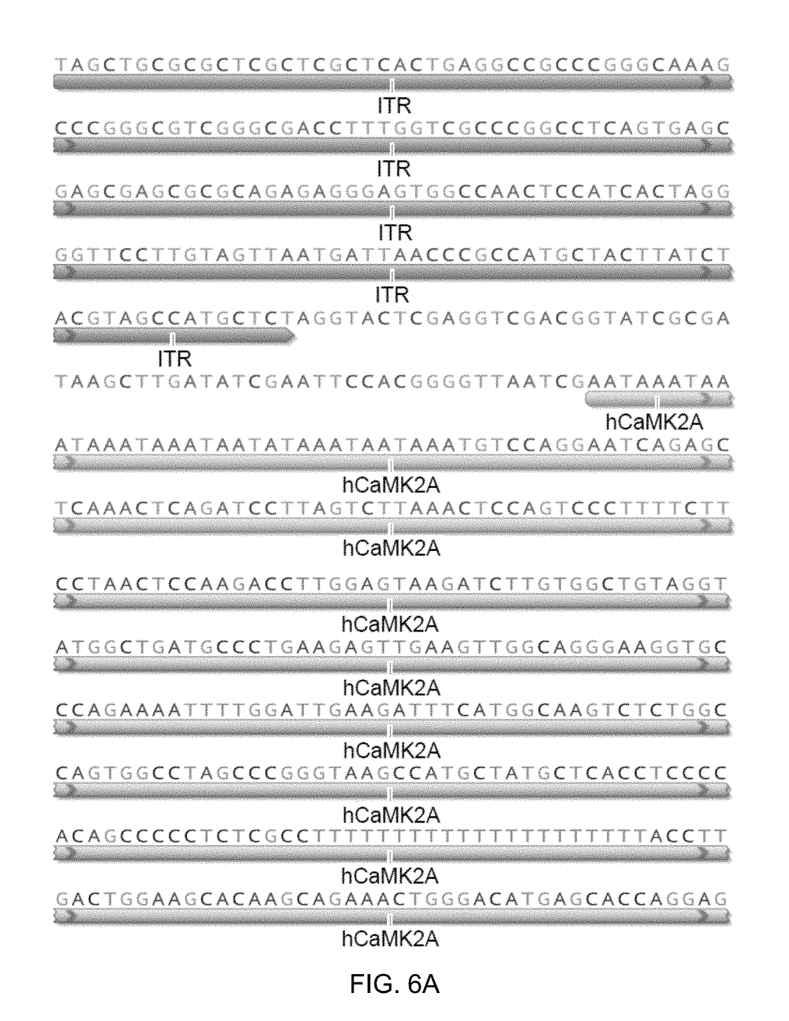

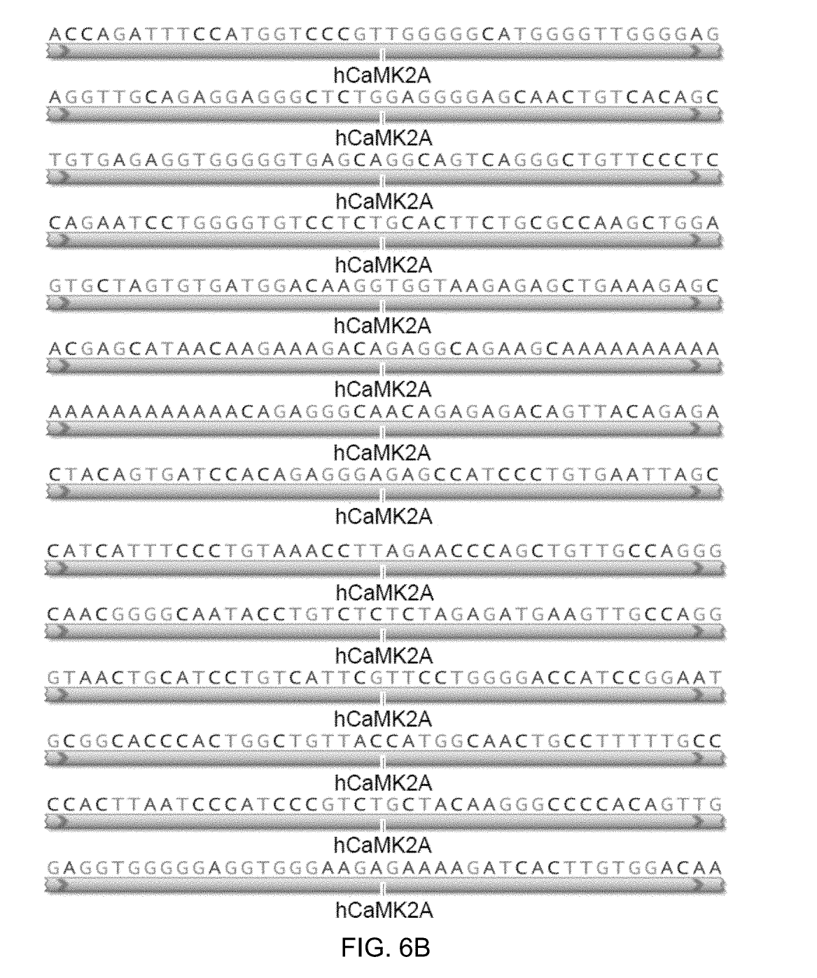

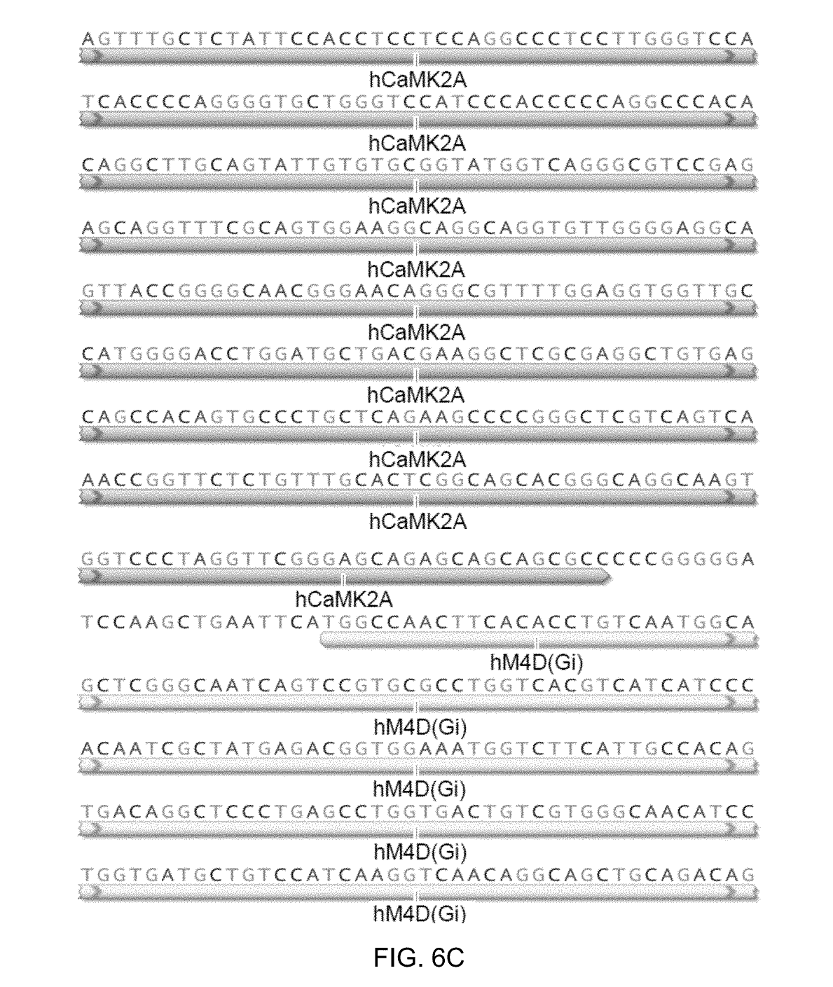

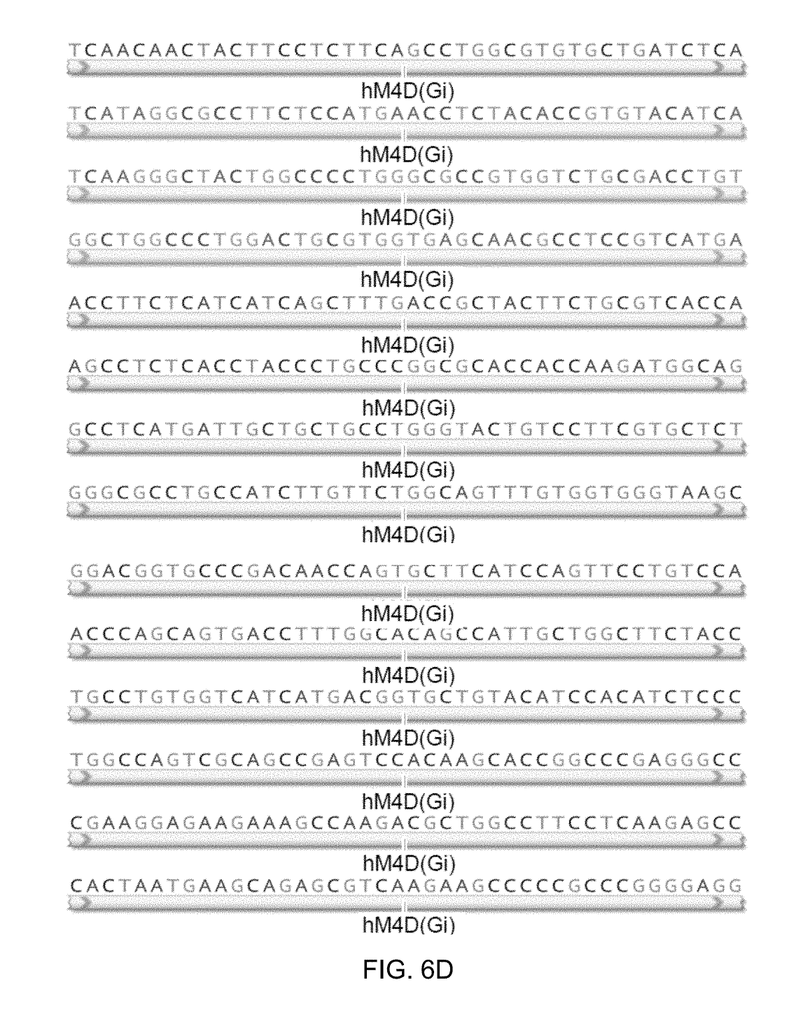

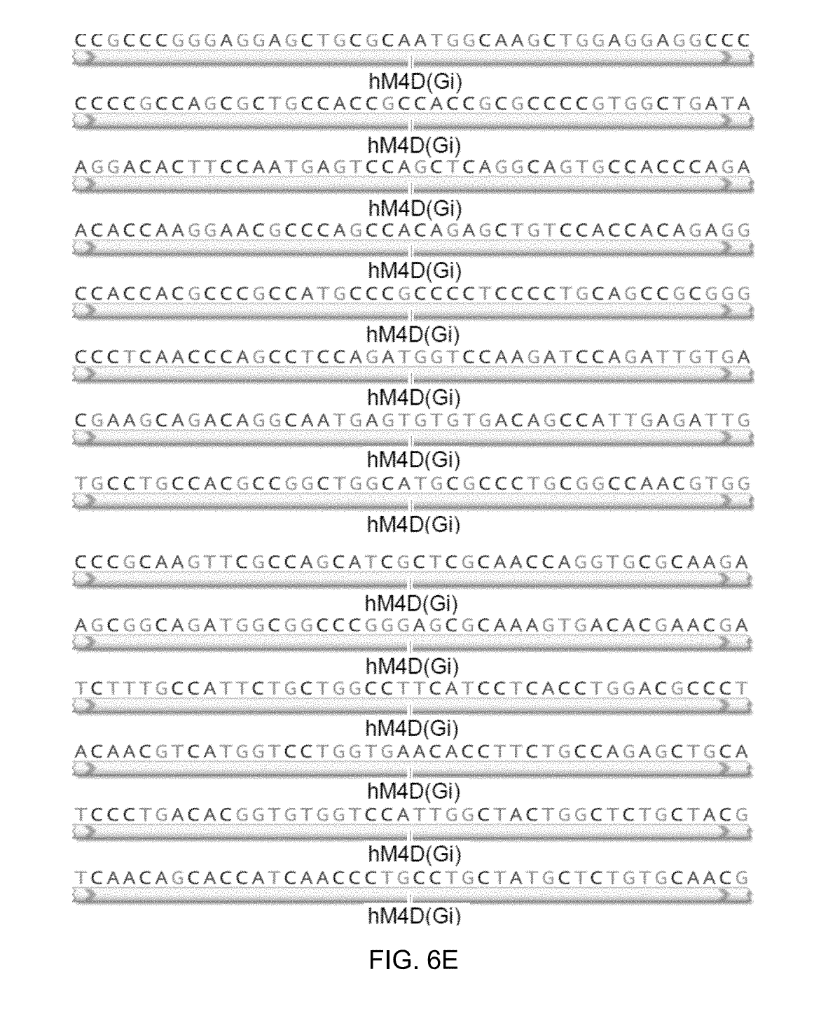

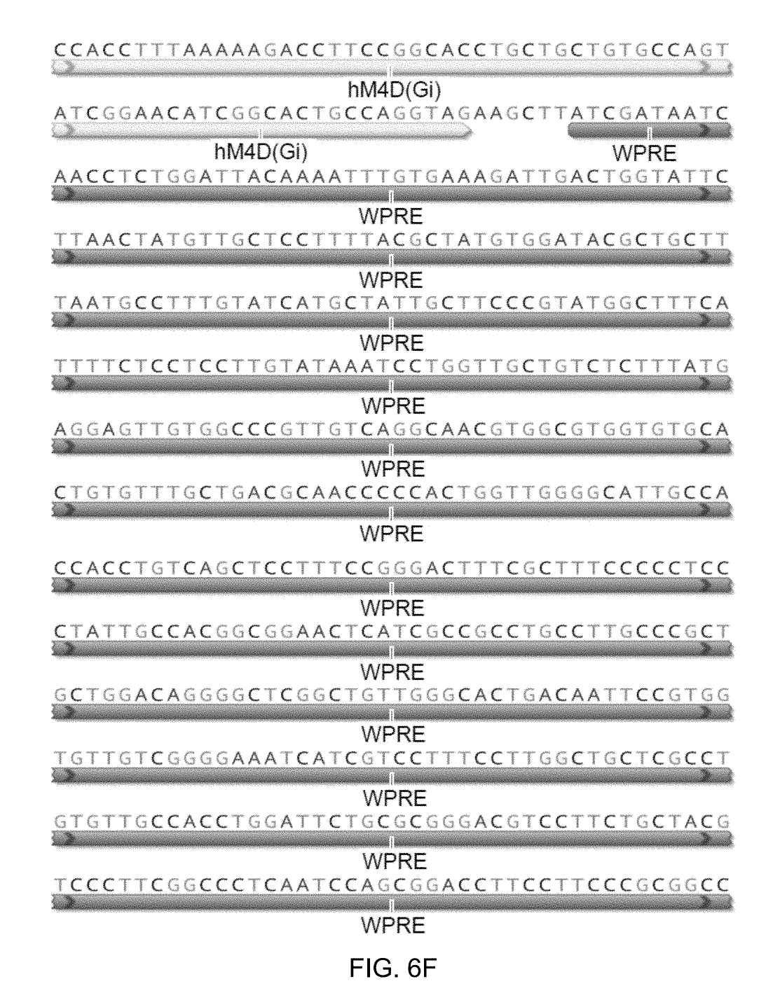

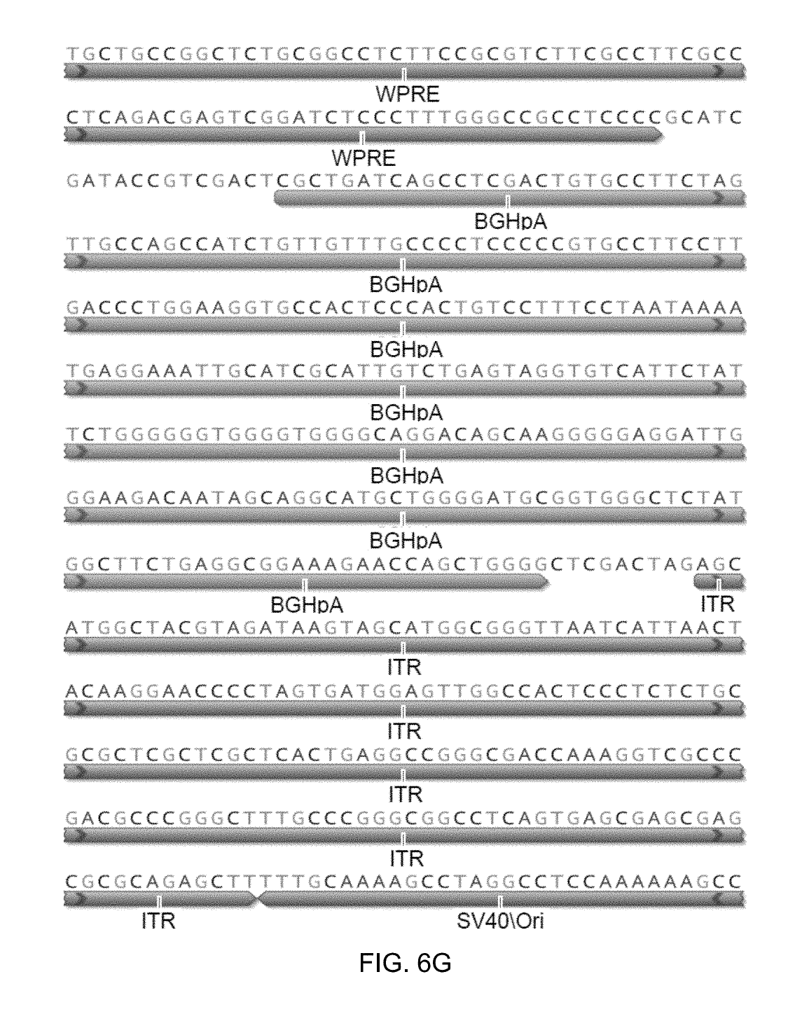

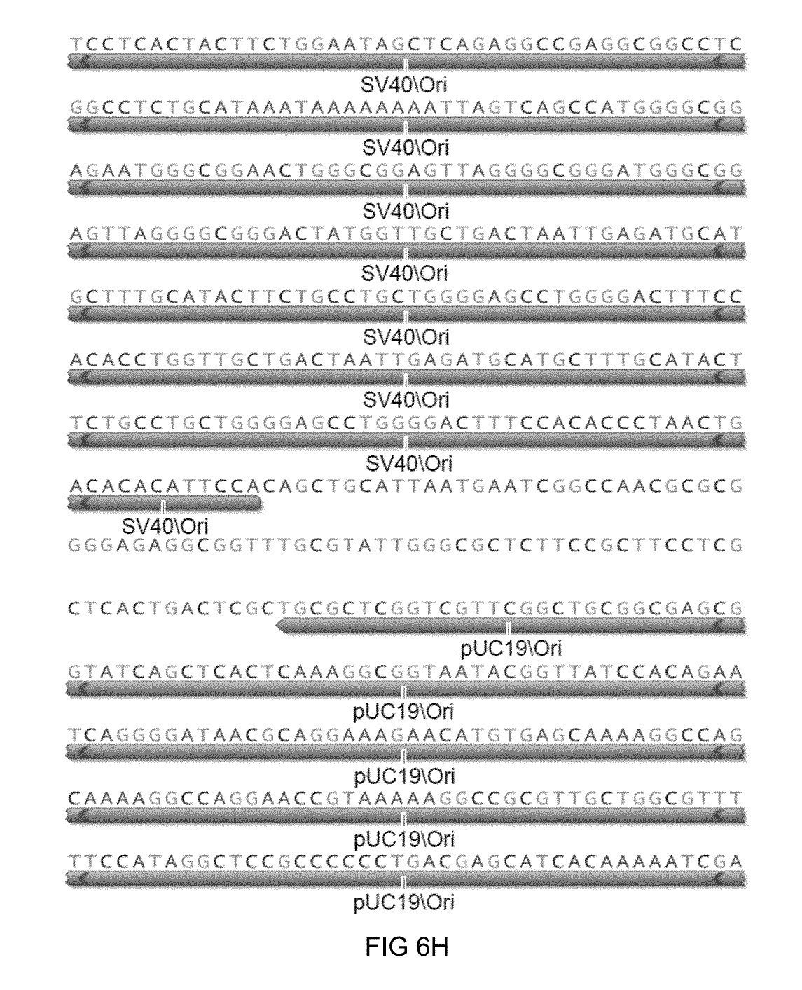

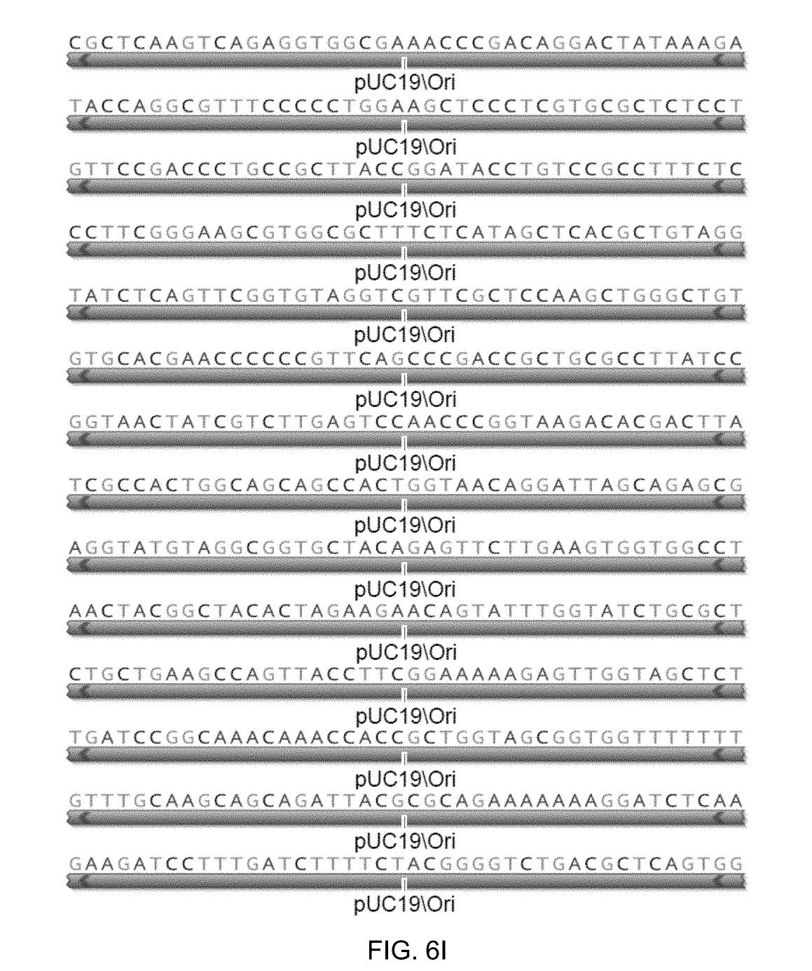

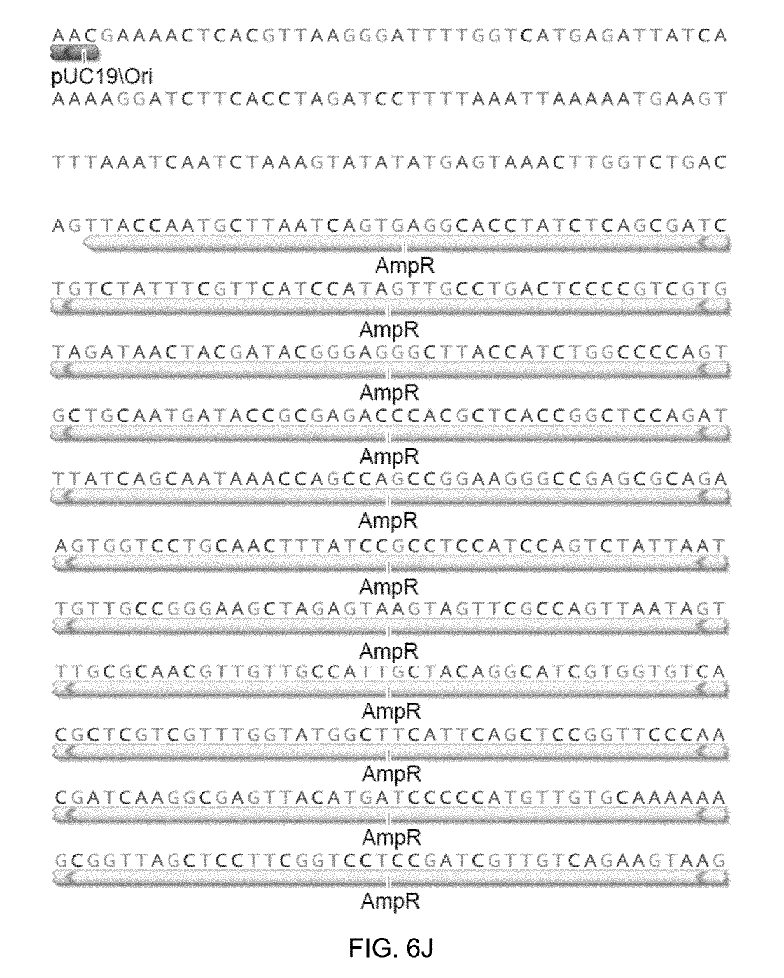

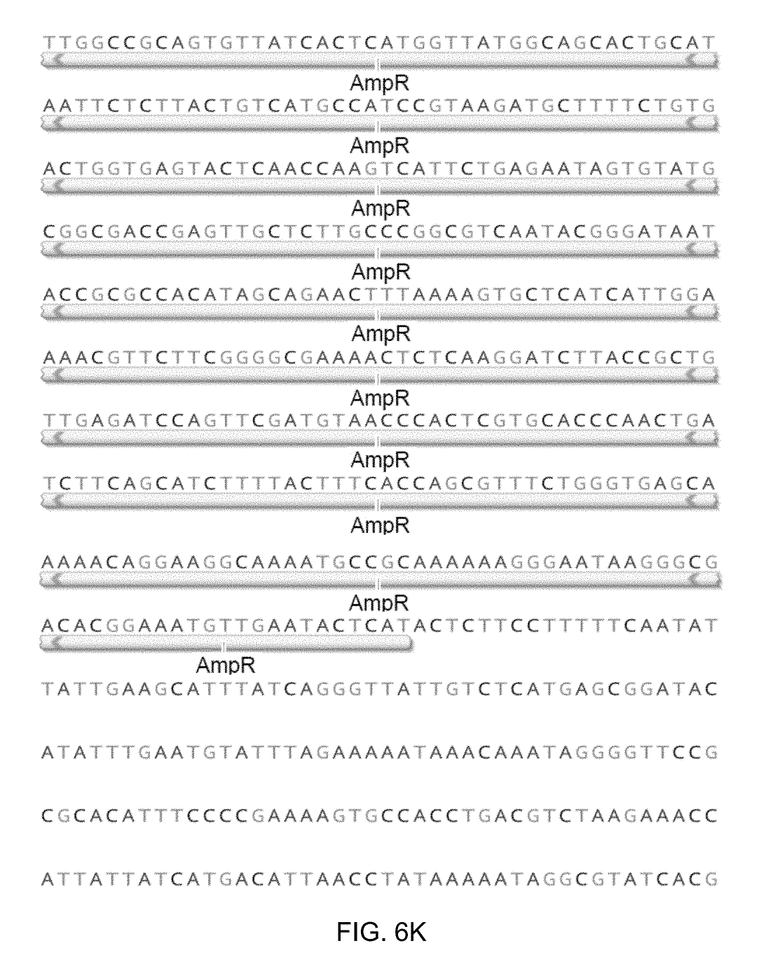

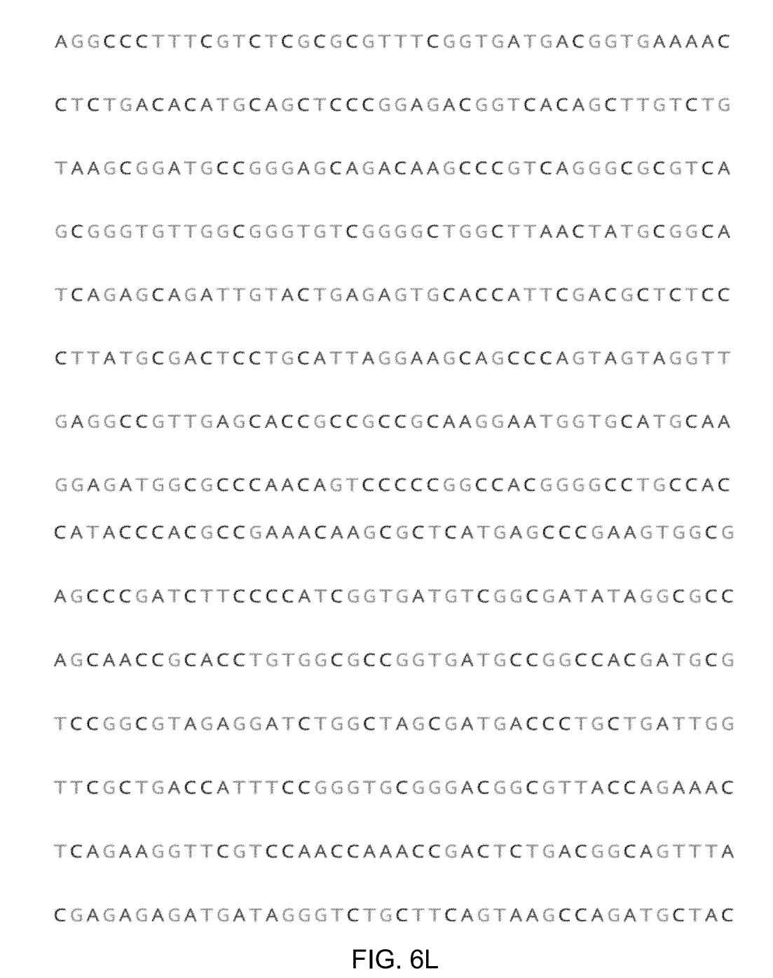

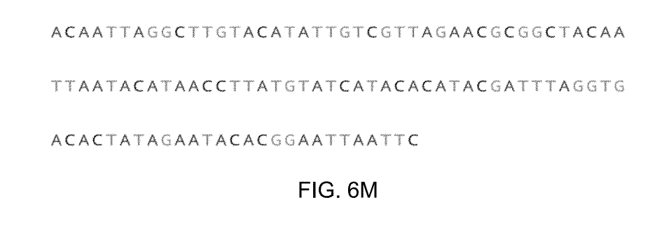

FIGS. 6A-6M are a schematic depiction showing correspondence between the location of components of pAM/hCaMK2A-hM4D(Gi)-WPRE-BGHpA and the nucleotide sequence.

The pAM/CaMKII-hM4D(Gi)-WPRE-BGHpA vector or the pAM/hCaMK2A-hM4D(Gi)-WPRE-BGHpA vector described herein along with synthetic ligands are used to treat seizure disorders. Seizure disorders, including those involving complex partial seizures, e.g., temporal lobe epilepsy (TLE) may be one of the most refractory forms of epilepsy. In certain instances, one temporal lobe may be defined as the site of seizure origin (the epileptogenic region) and the medial temporal lobe including the anterior hippocampus may be targeted in accordance with the methods herein. Seizure disorders can result from an imbalance of excitation to inhibition. The antagonism of excitation and enhancing of inhibition can provide improvement in at least one symptom of the seizure disorder.

Examples of seizure disorders include epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, brain tumor induced seizures, hamartoma induced seizures, drug withdrawal induced seizures, alcohol withdrawal induced seizures, increased seizure activity or breakthrough seizures (also called serial or cluster seizures). In embodiments, the seizure disorder is associated with a sodium channel protein type 1 subunit alpha (Scn1a)-related disorder. In embodiments, the seizure disorder is characterized by focal seizures. In embodiments, the seizure disorder is focal cortical dysplasia. In embodiments, the FCD is Type I FCD. In embodiments, the FCD is Type Ia FCD. In embodiments, the FCD is Type Ib FCD. In embodiments, the FCD is Type Ic FCD. In embodiments, the FCD is Type II FCD. In embodiments, the FCD is Type IIa FCD. In embodiments, the FCD is Type IIb FCD. In embodiments, the FCD is Type III FCD. In embodiments, the FCD is Type IIIa FCD. In embodiments, the FCD is Type IIIb FCD. In embodiments, the FCD is Type IIIc FCD. In embodiments, the seizure disorder is associated with a brain tumor, i.e., brain tumor induced seizures, such as a ganglioglioma, a glioma—low grade and high grade, including anaplastic astrocytoma, anaplastic oligodendroglioma, anaplastic oligoastrocytoma, and anaplastic ependymoma, a glioblastoma, or a meningioma. In embodiments, the seizure disorder is associated with brain hamartomas, i.e., hamartoma induced seizures, such as Tuberous Sclerosis Complex (TSC) or Tuber Cinereum Hamartoma.

In embodiments, the seizure disorder is status epilepticus (SE). SE is characterized by an epileptic seizure of greater than five minutes or more than one seizure within a five-minute period without the person returning to normal between them. SE can be a dangerous condition that can lead to mortality if treatment is delayed. SE can be convulsive, with a regular pattern of contraction and extension of the arms and legs, or non-convulsive, with a change in a person's level of consciousness of relatively long duration but without large scale bending and extension of the limbs due to seizure activity. Convulsive SE (CSE) may be further classified into (a) tonic-clonic SE, (b) tonic SE, (c) clonic SE and (d) myoclonic SE. Non-convulsive SE (NCSE) is characterized by abnormal mental status, unresponsiveness, ocular motor abnormalities, persistent electrographic seizures, and possible response to anticonvulsants.

Symptoms of a seizure disorder may include, but are not limited to, episodes involving ataxia, gait impairment, speech impairment, vocalization, involuntary laughter, impaired cognition, abnormal motor activity, clinical seizure, subclinical seizure, hypotonia, hypertonia, drooling, mouthing behavior, aura, repetitive movements, and unusual sensations. In embodiments, the methods and compositions provided may reduce or prevent one or more different types of seizures. Generally, a seizure can include repetitive movements, unusual sensations, and combinations thereof. Seizures can be categorized as focal seizures (also referred to as partial seizures) and generalized seizures. Focal seizures affect only one side of the brain, while generalized seizures affect both sides of the brain. Specific types of focal seizures include simple focal seizures, complex focal seizures, and secondarily generalized seizures. Simple focal seizures can be restricted or focused on a particular lobe (e.g., temporal lobe, frontal lobe, parietal lobe, or occipital lobe). Complex focal seizures generally affect a larger part of one hemisphere than simple focal seizures, but commonly originate in the temporal lobe or the frontal lobe. When a focal seizure spreads from one side (hemisphere) to both sides of the brain, the seizure is referred to as a secondarily generalized seizure. Specific types of generalized seizures include absences (also referred to as petit mal seizures), tonic seizures, atonic seizures, myoclonic seizures, tonic clonic seizures (also referred to as grand mal seizures), and clonic seizures. Methods of treatment herein can include providing improvement in one or more of the foregoing symptoms.

Once a determination has been made of the location or of a suspected location of abnormal electrical impulses associated with a seizure disorder in a patient, targeted treatment in accordance with the present disclosure can be implemented. Methods of determining the location of abnormal electrical activity in the brain are well-known in the art. Although any area exhibiting abnormal electricity in the brain can be targeted for treatment herein, areas of the brain which are known to be associated with seizure disorders and which can receive targeted treatment include, but are not limited to, the temporal lobe, the frontal lobe, the occipital lobe and the parietal lobe. For example, the temporal lobes can be a common site of localized epileptic seizures. In certain instances, seizures beginning in the temporal lobes can extend to other parts of the brain. In embodiments, specific areas of the temporal lobe which can be targeted for treatment include structures of the limbic system such as the hippocampus, auditory-vestibular cortex, the medial temporal lobe, and the amygdala. In embodiments, specific areas of the occipital lobe can also be targeted, e.g., the primary visual cortex. In embodiments, specific areas of the parietal lobe can be targeted, e.g., the lateral postcentral gyms. In embodiments, the location of the primary somatosensory cortex which can be targeted. In embodiments, specific areas of the frontal lobe can be targeted, e.g., the motor cortex, the olfactory-gustatory cortex. In embodiments, large areas of the brain which have been identified as exhibiting abnormal electrical activity can be targeted. In certain instances, manifestations of seizure disorders can begin within certain areas of the brain and spread to others. For example, manifestations of seizure disorders can begin within the hippocampus or its surrounding structures. In embodiments, areas determined to be the site of origin of the abnormal electrical activity can be targeted.

Methods for administering materials directly to target locations within the brain are well-known. For example, a hole, e.g., Burr hole, can be drilled into the skull and an appropriately sized needle may be used to deliver a vector to a target location. In embodiments, a portion of the skull may be removed to expose the dura matter (craniotomy) at or near a target location and a vector can be administered directly to the target location. In embodiments, a vector is injected intracranially using stereotaxic coordinates, a micropipette and an automated pump for precise delivery of the vector to the desired area with minimal damage to the surrounding tissue. In embodiments, a micropump may be utilized to deliver pharmaceutical compositions containing a vector encoding hD4Mi to target areas in the brain. The compositions can be delivered immediately or over an extended period of time, e.g., over 1, 2, 3, 4, 5, 6, 7, 8, 9, 10 or more minutes. After vector delivery to a target location in the brain a sufficient amount of time may be allowed to pass to allow expression of hD4Mi at the target location.

In embodiments, pharmaceutical compositions containing vectors herein can be administered systemically. Systemic delivery includes oral, buccal, sublingual, rectal, topical, intranasal, vaginal and parenteral modes of administration. Examples of parenteral modes of administration include intravenous, intraperitoneal, intramuscular and subcutaneous modes of administration. In embodiments, the vector will circulate until it contacts the target location(s) in the brain where it delivers nucleic acid encoding hD4Mi and causes hD4Mi to be expressed and act, e.g., to modulate neuronal signaling networks, when activated by synthetic ligands.

As previously mentioned, synthetic ligands herein include clozapine N-oxide (CNO), perlapine. CNO may also referred to as 8-chloro-11-(4-methyl-1-piperazinyl)-5H-dibenzo[b,e](1,4)diazepine N-oxide. In embodiments, CNO can be administered directly to a target location in the brain by any known means for administering materials to the brain, e.g., direct injection. In embodiments, CNO can be administered systemically to the patient. Systemic delivery includes oral, buccal, sublingual, rectal, topical, intranasal, vaginal and parenteral modes of administration. Examples of parenteral modes of administration include intravenous, intraperitoneal, intramuscular and subcutaneous modes of administration.

Suitable effective CNO dosages may vary based on DREADD receptor expression levels, cell types infected, duration of DREADD activation desired, and the species being treated. In embodiments, CNO administration can range from 0.1-50 mg/kg. In embodiments, pharmaceutical compositions containing CNO for treating a seizure disorder may include CNO in an amount of, e.g., about 0.01 to 500 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 0.5 to 500 mg, 0.5 to 450 mg, 0.5 to 300 mg, 0.5 to 250 mg, 0.5 to 200 mg, 0.5 to 175 mg, 0.5 to 150 mg, 0.5 to 125 mg, 0.5 to 100 mg, 0.5 to 75 mg, 0.5 to 50 mg, 0.5 to 30 mg, 0.5 to 25 mg, 0.5 to 20 mg, 0.5 to 15 mg, 0.5 to 10 mg, 0.5 to 5 mg, 0.5 to lmg, 1 to 500 mg, 1 to 450 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 500 mg, 5 to 450 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 500 mg, 10 to 450 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 500 mg, 15 to 450 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 500 mg, 20 to 450 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 500 mg, 25 to 450 mg, 25 to 300 mg, 25 to 250 mg, 25 to 200 mg, 25 to 175 mg, 25 to 150 mg, 25 to 125 mg, 25 to 100 mg, 25 to 80 mg, 25 to 75 mg, 25 to 50 mg, 25 to 30 mg, 30 to 500 mg, 30 to 450 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 40 to 500 mg, 40 to 450 mg, 40 to 400 mg, 40 to 250 mg, 40 to 200 mg, 40 to 175 mg, 40 to 150 mg, 40 to 125 mg, 40 to 100 mg, 40 to 75 mg, 40 to 50 mg, 50 to 500 mg, 50 to 450 mg, 50 to 300 mg, 50 to 250 mg, 50 to 200 mg, 50 to 175 mg, 50 to 150 mg, 50 to 125 mg, 50 to 100 mg, 50 to 75 mg, 75 to 500 mg, 75 to 450 mg, 75 to 300 mg, 75 to 250 mg, 75 to 200 mg, 75 to 175 mg, 75 to 150 mg, 75 to 125 mg, 75 to 100 mg, 100 to 500 mg, 100 to 450 mg, 100 to 300 mg, 100 to 250 mg, 100 to 200 mg, 100 to 175 mg, 100 to 150 mg, 100 to 125 mg, 125 to 500 mg, 125 to 450 mg, 125 to 300 mg, 125 to 250 mg, 125 to 200 mg, 125 to 175 mg, 125 to 150 mg, 150 to 500 mg, 150 to 450 mg, 150 to 300 mg, 150 to 250 mg, 150 to 200 mg, 200 to 500 mg, 200 to 450 mg, 200 to 300 mg, 200 to 250 mg, 250 to 500 mg, 250 to 450 mg, 250 to 300 mg, 300 to 500 mg, 300 to 450 mg, 300 to 400 mg, 300 to 350 mg, 350 to 500 mg, 350 to 450 mg, 350 to 400 mg, 400 to 500 mg, 400 to 450 mg, with 0.1 mg, 0.25 mg, 0.5 mg, 0.75 mg, 1 mg, 2.5 mg, 5 mg, 7.5 mg, 10 mg, 12.5 mg, 15 mg, 17.5 mg, 20 mg, 22.5 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg 175 mg, 200 mg, 225 mg, 250 mg, 275 mg, 300 mg, 325 mg, 350 mg, 375 mg, 400 mg, 425 mg, 450 mg, 475 mg, and 500 mg being examples. Suitable dosages of CNO may be administered to a patient having a seizure disorder once, twice, three, four times, five or six times daily, every other day, once weekly, or once a month.

In embodiments CNO is administered to a patient having a seizure disorder twice a day, (e.g., morning and evening), or three times a day (e.g., at morning, noon, and bedtime), at a dose of 0.1-200 mg/administration. In embodiments, CNO is administered to a patient having a seizure disorder 1000 mg/per day, 600 mg/day, 550 mg/per day, 500 mg/per day, 450 mg/per day, 400 mg/per day, 350 mg/per day, 300 mg/per day, 250 mg/per day, 225 mg/per day, 200 mg/per day, 190 mg/per day, 180 mg/per day, 170 mg/per day, 160 mg/per day, 150 mg/per day, 140 mg/per day, 130 mg/per day, 120 mg/per day, 110 mg/per day, 100 mg/per day, 95 mg/per day, 90 mg/per day, 85 mg/per day, 80 mg/per day, 75 mg/per day, 70 mg/per day, 65 mg/per day, 60 mg/per day, 55 mg/per day, 50 mg/per day, 45 mg/per day, 40 mg/per day, 35 mg/per day, 30 mg/per day, 25 mg/per day, 20 mg/per day, 15 mg/per day, 10 mg/per day, 5 mg/per day, 4 mg/per day, 3 mg/per day, 3 mg/per day, 2 mg/per day, 1 mg/per day, in one or more doses. In embodiments, an adult dose can be about 0.05 to 500 mg per day and can be increased to 750 mg per day. Dosages can be lower for infants and children than for adults. In embodiments, an infant or pediatric dose can be about 0.1 to 50 mg per day once or in 2, 3 or 4 divided doses. In embodiments, a pediatric dose can be 0.75 mg/kg/day to 1.5 mg/kg/day. In embodiments, the patient may be started at a low dose and the dosage is escalated over time.

Perlapine may also be administered as a synthetic ligand. Perlapine may also referred to as 6-(4-Methyl-1-piperazinyl)-11H-dibenz[b,e]azepine, or 6-(4-methylpiperazin-1-yl) morphanthridine. In embodiments, perlapine can be administered directly to a target location in the brain by any known means for administering materials to the brain, e.g., direct injection. In embodiments, perlapine can be administered systemically to the patient. Systemic delivery includes oral, buccal, sublingual, rectal, topical, intranasal, vaginal and parenteral modes of administration. Examples of parenteral modes of administration include intravenous, intraperitoneal, intramuscular and subcutaneous modes of administration.

Suitable effective perlapine dosages may vary based on DREADD receptor expression levels, cell types infected, duration of DREADD activation desired, and the species being treated. In embodiments, perlapine administration can range from 0.01-1 mg/kg.

In embodiments, pharmaceutical compositions containing perlapine for treating a seizure disorder may include perlapine in an amount of, e.g., 0.1 mg to 75 mg, 0.1 mg to 70 mg, 0.1 mg to 65 mg, 0.1 mg to 55 mg, 0.1 mg to 50 mg, 0.1 mg to 45 mg, 0.1 mg to 40 mg, 0.1 mg to 35 mg, 0.1 mg to 30 mg, 0.1 mg to 25 mg, 0.1 mg to 20 mg, 0.1 mg to 15 mg, 0.1 mg to 10 mg, 0.5 mg to 75 mg, 0.5 mg to 70 mg, 0.5 mg to 65 mg, 0.5 mg to 55 mg, 0.5 mg to 50 mg, 0.5 mg to 45 mg, 0.5 mg to 40 mg, 0.5 mg to 35 mg, 0.5 mg to 30 mg, 0.5 mg to 25 mg, 0.5 mg to 20 mg, 0.5 to 15 mg, 0.5 to 10 mg, 1 mg to 75 mg, 1 mg to 70 mg, 1 mg to 65 mg, 1 mg to 55 mg, 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1.5 mg to 75 mg, 1.5 mg to 70 mg, 1.5 mg to 65 mg, 1.5 mg to 55 mg, 1.5 mg to 50 mg, 1.5 mg to 45 mg, 1.5 mg to 40 mg, 1.5 mg to 35 mg, 1.5 mg to 30 mg, 1.5 mg to 25 mg, 1.5 mg to 20 mg, 1.5 mg to 15 mg, 1.5 mg to 10 mg, 2 mg to 75 mg, 2 mg to 70 mg, 2 mg to 65 mg, 2 mg to 55 mg, 2 mg to 50 mg, 2 mg to 45 mg, 2 mg to 40 mg, 2 mg to 35 mg, 2 mg to 30 mg, 2 mg to 25 mg, 2 mg to 20 mg, 2 mg to 15 mg, 2 mg to 10 mg, 2.5 mg to 75 mg, 2.5 mg to 70 mg, 2.5 mg to 65 mg, 2.5 mg to 55 mg, 2.5 mg to 50 mg, 2.5 mg to 45 mg, 2.5 mg to 40 mg, 2.5 mg to 35 mg, 2.5 mg to 30 mg, 2.5 mg to 25 mg, 2.5 mg to 20 mg, 2.5 mg to 15 mg, 2.5 mg to 10 mg, 3 mg to 75 mg, 3 mg to 70 mg, 3 mg to 65 mg, 3 mg to 55 mg, 3 mg to 50 mg, 3 mg to 45 mg, 3 mg to 40 mg, 3 mg to 35 mg, 3 mg to 30 mg, 3 mg to 25 mg, 3 mg to 20 mg, 3 mg to 15 mg, 3 mg to 10 mg, 3.5 mg to 75 mg, 3.5 mg to 70 mg, 3.5 mg to 65 mg, 3.5 mg to 55 mg, 3.5 mg to 50 mg, 3.5 mg to 45 mg, 3.5 mg to 40 mg, 3.5 mg to 35 mg, 3.5 mg to 30 mg, 3.5 mg to 25 mg, 3.5 mg to 20 mg, 3.5 mg to 15 mg, 3.5 mg to 10 mg, 4 mg to 75 mg, 4 mg to 70 mg, 4 mg to 65 mg, 4 mg to 55 mg, 4 mg to 50 mg, 4 mg to 45 mg, 4 mg to 40 mg, 4 mg to 35 mg, 4 mg to 30 mg, 4 mg to 25 mg, 4 mg to 20 mg, 4 mg to 15 mg, 4 mg to 10 mg, 4.5 mg to 75 mg, 4.5 mg to 70 mg, 4.5 mg to 65 mg, 4.5 mg to 55 mg, 4.5 mg to 50 mg, 4.5 mg to 45 mg, 4.5 mg to 40 mg, 4.5 mg to 35 mg, 4.5 mg to 30 mg, 4.5 mg to 25 mg, 4.5 mg to 20 mg, 4.5 mg to 15 mg, 4.5 mg to 10 mg, 5 mg to 75 mg, 5 mg to 70 mg, 5 mg to 65 mg, 5 mg to 55 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, or 5 mg to 10 mg, perlapine or a pharmaceutically acceptable salt thereof.

In embodiments, pharmaceutical compositions include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg perlapine or a pharmaceutically acceptable salt thereof.

In embodiments, pharmaceutical compositions include 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, or 20 mg perlapine or a pharmaceutically acceptable salt thereof or amounts that are multiples of such doses. In embodiments, pharmaceutical compositions include 2.5 mg, 5 mg, 7.5 mg, 10 mg, 15 mg, 20 mg, 25 mg, 30 mg, 35 mg, or 40 mg perlapine or a pharmaceutically acceptable salt thereof.

In embodiments, compositions include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 7 mg, 7.5 mg, 10 mg, 12.5 mg, 15 mg, 17.5 mg, or 20 mg perlapine or a pharmaceutically acceptable salt thereof or amounts that are multiples of such doses.

In embodiments, pharmaceutical compositions include from about 0.05 mg to about 100 mg perlapine or a pharmaceutically acceptable salt thereof. In embodiments, dosage forms include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg perlapine or a pharmaceutically acceptable salt thereof.

Suitable dosages of perlapine may be administered to a patient having a seizure disorder once, twice, three, four times, five or six times daily, every other day, once weekly, or once a month. In embodiments perlapine is administered to a patient having a seizure disorder twice a day, (e.g., morning and evening), or three times a day (e.g., at morning, noon, and bedtime), at a dose of 0.1-50 mg/administration. In embodiments, perlapine is administered to a patient having a seizure disorder 250 mg/day, 190 mg/per day, 180 mg/per day, 170 mg/per day, 160 mg/per day, 150 mg/per day, 140 mg/per day, 130 mg/per day, 120 mg/per day, 110 mg/per day, 100 mg/per day, 95 mg/per day, 90 mg/per day, 85 mg/per day, 80 mg/per day, 75 mg/per day, 70 mg/per day, 65 mg/per day, 60 mg/per day, 55 mg/per day, 50 mg/per day, 45 mg/per day, 40 mg/per day, 35 mg/per day, 30 mg/per day, 25 mg/per day, 20 mg/per day, 15 mg/per day, 10 mg/per day, 5 mg/per day, 4 mg/per day, 3 mg/per day, 3 mg/per day, 2 mg/per day, 1 mg/per day, in one or more doses. In embodiments, an adult dose can be about 0.05 to 100 mg per day and can be increased to 200 mg per day. Dosages can be lower for infants and children than for adults. In embodiments, an infant or pediatric dose can be about 0.1 to 20 mg per day once or in 2, 3 or 4 divided doses. In embodiments, a pediatric dose can be 0.75 mg/kg/day to 1.5 mg/kg/day. In embodiments, the patient may be started at a low dose and the dosage is escalated over time.

In embodiments, methods of treating a seizure disorder are provided which include administering to the patient a vector encoding a modified receptor for delivery of the modified receptor to the target location, the modified receptor being modified to be activated by a synthetic ligand, wherein the modified receptor inhibits neuronal firing when activated; and administering to the patient the synthetic ligand, wherein the treatment provides improvement in one or more symptoms of the disorder for more than 1 hour after administration to the patient. In embodiments, the treatment provides improvement in one or more symptoms of the disorder for more than 2 hours after administration of the synthetic ligand to the patient. In embodiments, the treatment provides improvement in one or more symptoms of the disorder for more than 3 hours after administration of the synthetic ligand to the patient. In embodiments, the treatment provides improvement in one or more symptoms of the disorder for more than 4 hours after administration of the synthetic ligand to the patient. In embodiments, the treatment provides improvement in one or more symptoms of the disorder for more than 6 hours after administration of the synthetic ligand to the patient. In embodiments, the treatment provides improvement in one or more symptoms of the disorder for more than 8, 10, 12, 14, 16, 18, 20, 22 or 24 hours after administration of the synthetic ligand to the patient. In embodiments, improvement in at least one symptom for 12 hours after administration of the synthetic ligand to the patient is provided in accordance with the present disclosure. In embodiments, the synthetic ligands provide improvement of next day functioning of the patient. For example, the synthetic ligand may provide improvement in one or more symptoms of the disorder for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep. In embodiments, provided herein are methods of treating a seizure disorder including administering to a patient in need thereof and who has received a modified receptor as described herein, a synthetic ligand, after a warning sign of an impending seizure is detected to reduce or prevent seizure activity.

In embodiments, the methods described herein are effective to reduce, delay, or prevent one or more other clinical symptoms of a seizure disorder. For example, the effect, in a patient having modified receptors in a target location of the brain, of a composition including a synthetic ligand or a pharmaceutically acceptable salt thereof, whose delivery is optionally enhanced by ultrasound energy on a particular symptom, pharmacologic, or physiologic indicator can be compared to an untreated patient, or the condition of the patient prior to treatment. In embodiments, the symptom, pharmacologic, and/or physiologic indicator is measured in a patient prior to treatment, and again one or more times after treatment is initiated. In embodiments, the control is a reference level, or average determined based on measuring the symptom, pharmacologic, or physiologic indicator in one or more patients that do not have the disease or condition to be treated (e.g., healthy patients). In embodiments, the effect of the treatment is compared to a conventional treatment that is within the purview of those skilled in the art.

Effective treatment of a seizure disorder (e.g., intractable focal seizures, focal cortical dysplasia, acute repetitive seizure, status epilepticus) herein may be established by showing reduction in the frequency or severity of symptoms (e.g., more than 10%, 20%, 30% 40% or 50%) after a period of time compared with baseline. For example, after a baseline period of 1 month, the patients having modified receptors may be randomly allocated a synthetic ligand or a pharmaceutically acceptable salt thereof, or placebo as add-on therapy to standard therapies, during a double-blind period of 2 months. Primary outcome measurements may include the percentage of responders on a synthetic ligand or a pharmaceutically acceptable salt thereof, and on placebo, defined as having experienced at least a 10% to 50% reduction of symptoms during the second month of the double-blind period compared with baseline.

In embodiments, pharmaceutical compositions containing synthetic ligands may be provided with conventional release or modified release profiles. Pharmaceutical compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers.

In embodiments, pharmaceutical compositions containing vectors encoding hM4Di and/or synthetic ligands are suitable for parenteral administration, including, e.g., intramuscular (i.m.), intravenous (i.v.), subcutaneous (s.c.), intraperitoneal (i.p.), or intrathecal (i.t.). Parenteral compositions include those intended for direct delivery of a vector to brain cells. Parenteral compositions must be sterile for administration by injection, infusion or implantation into the body and may be packaged in either single-dose or multi-dose containers. In embodiments, liquid pharmaceutical compositions for parenteral administration to a patient include an active substance, e.g., vectors and/or synthetic ligands or a pharmaceutically acceptable salt of the synthetic ligands, in any of the respective amounts described above. In embodiments, the pharmaceutical compositions for parenteral administration are formulated as a total volume of about, e.g., 0.1 ml, 0.25 ml, 0.5 ml, 0.75 ml, 1 ml, 1.25 ml, 1.5 ml, 1.75 ml, 2 ml, 2.25 ml, 2.5 ml, 2.75 ml, 3 ml, 3.25 ml, 3.5 ml, 3.75 ml, 4 ml, 4.25 ml, 4.5 ml, 4.75 ml, 5 ml, 10 ml, 20 ml, 25 ml, 50 ml, 100 ml, 200 ml, 250 ml, or 500 ml. In embodiments, the volume of pharmaceutical compositions containing vectors are microliter amounts. For example, 0.1 microliters to 10 or more microliters can be injected. For example, 0.1, 0.2, 0.3, 0.4, 0.5, 0.6, 0.7, 0.8, 0.9, 1.0, 1.25, 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 8.25, 8.5, 8.75, 9.0, 9.25, 9.5, 9.75, or 10 microliters. In embodiments, the compositions are contained in a micropipette, a bag, a glass vial, a plastic vial, or a bottle.

In embodiments, pharmaceutical compositions for parenteral administration include respective amounts described above for the synthetic ligands or a pharmaceutically acceptable salt thereof. In embodiments, pharmaceutical compositions for parenteral administration include about 0.0001 mg to about 500 mg active substance, e.g., vectors or a synthetic ligand or a pharmaceutically acceptable salt of the synthetic ligand. In embodiments, pharmaceutical compositions for parenteral administration to a patient include an active substance, e.g., vectors or a synthetic ligand or a pharmaceutically acceptable salt of the synthetic ligand, at a respective concentration of about 0.001 mg/ml to about 500 mg/ml. In embodiments, the pharmaceutical composition for parenteral administration includes an active substance, vector or a synthetic ligand or a pharmaceutically acceptable salt of the synthetic ligand, at a respective concentration of, e.g., about 0.005 mg/ml to about 50 mg/ml, about 0.01 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the pharmaceutical composition for parenteral administration includes an active substance, e.g., vector or a synthetic ligand or a pharmaceutically acceptable salt of the synthetic ligand, at a respective concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml.

In embodiments, a pharmaceutical composition for parenteral administration is provided wherein the pharmaceutical composition is stable for at least six months. In embodiments, the pharmaceutical compositions for parenteral administration exhibit no more than about 5% decrease in active substance, e.g., vector or a synthetic ligand or a pharmaceutically acceptable salt of the synthetic ligand, for at least, e.g., 3 months or 6 months. In embodiments, the amount of vector or a synthetic ligand or a pharmaceutically acceptable salt of the synthetic ligand, degrades at no more than about, e.g., 2.5%, 1%, 0.5% or 0.1%. In embodiments, the degradation is less than about, e.g., 5%, 2.5%, 1%, 0.5%, 0.25%, 0.1%, for at least six months.

In embodiments, pharmaceutical compositions for parenteral administration are provided wherein the pharmaceutical composition remains soluble. In embodiments, pharmaceutical compositions for parenteral administration are provided that are stable, soluble, local site compatible and/or ready-to-use. In embodiments, the pharmaceutical compositions herein are ready-to-use for direct administration to a patient in need thereof.

The pharmaceutical compositions for parenteral administration provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of a vector, a synthetic ligand or a pharmaceutically acceptable salt of the synthetic ligand, used in the composition. Thus, parenteral compositions are provided wherein there is no incompatibility between any of the components of the dosage form.

In embodiments, parenteral compositions including a vector or a synthetic ligand or a pharmaceutically acceptable salt of the synthetic ligand, include a stabilizing amount of at least one excipient. For example, excipients may be selected from the group consisting of buffering agents, solubilizing agents, tonicity agents, antioxidants, chelating agents, antimicrobial agents, and preservative. One skilled in the art will appreciate that an excipient may have more than one function and be classified in one or more defined group.

In embodiments, parenteral compositions include a vector or a synthetic ligand or a pharmaceutically acceptable salt of the synthetic ligand, and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions may be administered as needed, e.g., once, twice, three, four, five, six or more times daily, or continuously depending on the patient's needs.

In embodiments, parenteral compositions of an active substance, e.g., a vector or a synthetic ligand or a pharmaceutically acceptable salt of the synthetic ligand, are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

It should be understood that dosage amounts of a synthetic ligand or a pharmaceutically acceptable salt of the synthetic ligand, that are provided herein are applicable to all the dosage forms described herein including conventional dosage forms, modified dosage forms, any first and second pharmaceutical compositions with differing release profiles, as well as the parenteral formulations described herein. Those skilled in the art will determine appropriate amounts of vectors and/or synthetic ligands depending on criteria such as dosage form, route of administration, patient tolerance, efficacy, therapeutic goal and therapeutic benefit, among other pharmaceutically acceptable criteria.

Combination therapies utilizing hM4Di, the synthetic ligands in combination with one or more AEDs are contemplated herein. Moreover, different pharmaceutical compositions having different release profiles, can include administration of the active agents together in the same admixture, or in separate admixtures.

In embodiments, provided herein are methods for treating a seizure disorder which include applying ultrasound to a target location in the patient's brain to enhance permeability of the patient's blood brain barrier at the target location; administering to the patient a vector encoding a modified receptor for delivery of the modified receptor to the target location, the modified receptor being modified to be activated by a synthetic ligand, wherein the modified receptor inhibits neuronal firing when activated; and administering to the patient the synthetic ligand wherein the patient exhibits improvement in at least one symptom of the seizure disorder.

In embodiments, treatment with ultrasound is used to enhance delivery of hM4Di to target locations in the brain by disrupting the blood brain barrier. Use of focused ultrasound energy herein disrupts the BBB without adversely affecting the vector, hM4Di, the synthetic ligand, and/or brain tissue itself. This may be considered surprising in view of potential damage to organic compounds and tissues by ultrasound energy. Use of ultrasound energy herein can increase the speed of delivery of vectors and/or synthetic ligands to target locations in the brain, reduce side effects which may be associated with delivery of vectors and/or synthetic ligands to target locations in the brain, reduce dosage amounts while concentrating vectors and/or synthetic ligands at a target location and can allow controlled release of the amount of vectors and/or synthetic ligands at a target location.

In accordance with the present disclosure, in embodiments, ultrasound energy assists and/or propels penetration of the vector carrying the modified receptor and/or synthetic ligand to target locations in the brain. In embodiments, ultrasound energy is used to make the blood brain barrier permeable to vectors herein. Accordingly, in embodiments, ultrasound energy can be applied to a target location prior to administration of the vector. In embodiments, vectors herein can be administered to a target area in the brain simultaneously with administration of ultrasound energy. In embodiments, vectors herein can be administered to a target area in the brain after administration of ultrasound energy.

As mentioned previously, vectors herein can be administered systemically. Systemic delivery includes oral, buccal, sublingual, rectal, topical, intranasal, vaginal and parenteral modes of administration. Examples of parenteral modes of administration include intravenous, intraperitoneal, intramuscular and subcutaneous modes of administration. In this manner, vectors circulating in the blood stream are delivered to a target location in the brain through a portion of the BBB disrupted by ultrasonic energy. In embodiments, vectors herein can be administered systemically after ultrasound energy treatment of the target location and the vectors penetrate the disrupted BBB to become situated at the target location. In embodiments, vectors herein can be administered directly to a target location in the brain. In embodiments, vectors herein can be administered directly to a target location in the brain after ultrasound energy treatment of the target location to become situated at the target location. In embodiments, vectors herein can be administered directly to a target location in the brain without ultrasound treatment.

In order to activate the modified receptor, a synthetic ligand which activates the modified receptor is administered to the patient. In embodiments, ultrasound energy is applied to a target area in the brain to disrupt the BBB to allow, assist and/or propel penetration of the synthetic ligand to the target location where it can interact with the modified receptor. In embodiments, the synthetic ligand can be administered directly to a target location in the brain by any known means for administering materials to the brain, e.g., direct injection through, e.g., a burr hole. In embodiments, the synthetic ligand can be administered systemically to the patient. Systemic delivery includes oral, buccal, sublingual, rectal, topical, intranasal, vaginal and parenteral modes of administration. Examples of parenteral modes of administration include intravenous, intraperitoneal, intramuscular and subcutaneous modes of administration. In this manner, synthetic ligands circulating in the blood stream are delivered to a target location in the brain through a portion of the BBB disrupted by ultrasound energy. In embodiments, ultrasound energy can be applied to a target area in the brain prior to administration of a synthetic ligand. In embodiments, ultrasound energy can be applied to a target area in the brain simultaneously with administration of a synthetic ligand. In embodiments, ultrasound energy can be applied to a target area in the brain after administration of a synthetic ligand.

In embodiments, ultrasound energy can be administered to a target area by removing a portion of the skull (craniotomy) to expose the dura matter at or near a target location and delivering the ultrasound energy at or below the exposed dura matter. In embodiments, ultrasound energy can be administered to a target location through the skull, eliminating the need for surgery associated with delivery of ultrasound energy to a target location. Methods for delivering ultrasound energy through the skull are known in the art. See, e.g., U.S. Pat. No. 5,752,515 and US Publication No. 2009/0005711, both of which are hereby incorporated by reference in their respective entireties. See also, Hynynen et al., NeuroImage 24 (2005) 12-120.

In embodiments, ultrasound energy can be applied to a target location in the brain at frequencies ranging from about 20 kHz to about 5 MHz, and with sonication duration ranging from 100 nanoseconds to 1 minute. In embodiments, ultrasound energy can be applied to a target location in the brain at frequencies ranging from about 20 kHz to about 10 MHz, sonication duration ranging from about 100 nanoseconds to about 30 minutes, with continuous wave or burst mode operation, where the burst mode repetition varies from about 0.01 Hz to about 1 MHz. In embodiments, ultrasound energy can be applied to a target location in the brain at frequencies ranging from about 200 kHz to about 10 MHz, and with sonication duration ranging from about 100 milliseconds to about 30 minutes. In embodiments, ultrasound energy can be applied to a target location in the brain at frequencies ranging from about 250 kHz to about 10 MHz, and with sonication duration ranging from about 0.10 microseconds to about 30 minutes. In embodiments, ultrasound energy can be applied to a target location in the brain at a frequency of about 1.525 MHz. In embodiments, ultrasound energy can be applied to a target location in the brain at a frequency of about 0.69 MHz. In embodiments, pressure amplitudes generated by ultrasound energy can be about 0.5 to about 2.7 MPa. In embodiments, pressure amplitudes generated by ultrasound energy can be about 0.8 to about 1 MPa. In embodiments, ultrasound energy is applied to a target location in the brain at a focal region sized in accord with the volume of tissue and/or fluids to which a vector or synthetic ligand is to be delivered, e.g., from about 0.1 mm³ to about 5 cm³.

In embodiments, the target location and access thereto is confirmed by introducing a contrast agent into the patient prior to, during or after application of ultrasound energy to the target location, allowing sufficient time for the contrast agent to permeate the BBB, and determining whether the contrast agent is present at the target location. Contrast agents are well-known and include, e.g., iodine-based compounds, barium-based compounds and lanthanide based compounds. Iodine-based agents include, e.g., iohexol, iopromide, iodixanol, iosimenol, ioxaglate, iothalamate and iopamidol. Barium-based compounds include barium sulfate. Lanthanide-based compounds include, e.g., gadolinium-based chelates such as gadoversetamide, gadopentetate dimeglumine, gadobutrol, gadobenate dimeglumine, gadoterate meglumine, and gadoxetate disodium. Detection modalities include 2-dimensional X-ray radiography, X-ray computed tomography and magnetic resonance imaging which are well-known techniques that may be utilized to confirm the presence or absence of contrast agent in a target location.

In embodiments, methods of treating a seizure disorder are provided which include applying ultrasound to a target location in the patient's brain to enhance permeability of the patient's blood brain barrier at the target location; administering to the patient a vector encoding a modified receptor for delivery of the modified receptor to the target location, the modified receptor being modified to be activated by a synthetic ligand, wherein the modified receptor inhibits neuronal firing when activated; and administering to the patient the synthetic ligand, wherein the treatment provides improvement in one or more symptoms of the disorder for more than 1 hour after administration to the patient. In embodiments, the treatment provides improvement in one or more symptoms of the disorder for more than 2 hours after administration of the synthetic ligand to the patient. In embodiments, the treatment provides improvement in one or more symptoms of the disorder for more than 3 hours after administration of the synthetic ligand to the patient. In embodiments, the treatment provides improvement in one or more symptoms of the disorder for more than 4 hours after administration of the synthetic ligand to the patient. In embodiments, the treatment provides improvement in one or more symptoms of the disorder for more than 6 hours after administration of the synthetic ligand to the patient. In embodiments, the treatment provides improvement in one or more symptoms of the disorder for more than 8, 10, 12, 14, 16, 18, 20, 22 or 24 hours after administration of the synthetic ligand to the patient. In embodiments, improvement in at least one symptom for 12 hours after administration of the synthetic ligand to the patient is provided in accordance with the present disclosure. In embodiments, the synthetic ligands provide improvement of next day functioning of the patient. For example, the synthetic ligand may provide improvement in one or more symptoms of the disorder for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

In embodiments, provided herein are methods of treating a seizure disorder including administering to a patient in need thereof and who has received a modified receptor as described herein, ultrasound energy and a synthetic ligand, after a warning sign of an impending seizure is detected to reduce or prevent seizure activity.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value. Alternatively, particularly with respect to biological systems or processes, the term can mean within an order of magnitude, preferably within 5-fold, and more preferably within 2-fold, of a value.

“Improvement” refers to the treatment of seizure disorders such as focal epilepsy, intractable focal epilepsy, focal cortical dysplasia, epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, brain tumor induced seizures, hamartoma induced seizures, drug withdrawal induced seizures, alcohol withdrawal induced seizures, increased seizure activity or breakthrough seizures (also called serial or cluster seizures), measured relative to at least one symptom of the foregoing disorders.

“Improvement in next day functioning” or “wherein there is improvement in next day functioning” refers to improvement after waking from an overnight sleep period wherein the beneficial effect of administration of one or more synthetic ligands to a patient applies to at least one symptom of a syndrome or disorder herein and is discernable, either subjectively by a patient or objectively by an observer, for a period of time, e.g., 2 hours, 3 hours, 4 hours, 5 hours, 6 hours, 12 hours, 24 hours, etc. after waking.

“Treating”, “treatment” or “treat” can refer to the following: alleviating or delaying the appearance of clinical symptoms of a disease or condition in a patient that may be afflicted with or predisposed to the disease or condition, but does not yet experience or display clinical or subclinical symptoms of the disease or condition. In certain embodiments, “treating”, “treat” or “treatment” may refer to preventing the appearance of clinical symptoms of a disease or condition in a patient that may be afflicted with or predisposed to the disease or condition, but does not yet experience or display clinical or subclinical symptoms of the disease or condition. “Treating”, “treat” or “treatment” also refers to inhibiting the disease or condition, e.g., arresting or reducing its development or at least one clinical or subclinical symptom thereof. “Treating”, “treat” or “treatment” further refers to relieving the disease or condition, e.g., causing regression of the disease or condition or at least one of its clinical or subclinical symptoms. The benefit to a patient to be treated may be statistically significant, mathematically significant, or at least perceptible to the patient and/or the physician. Nonetheless, prophylactic (preventive) treatment and therapeutic (curative) treatment are two separate embodiments of the disclosure herein.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount” can mean a dosage sufficient to alleviate one or more symptoms of a syndrome, disorder, disease, or condition being treated, or to otherwise provide a desired pharmacological and/or physiologic effect. “Effective amount” or “therapeutically effective amount” may be used interchangeably herein.

“Co-administered with”, “co-administration with”, “administered in combination with”, “a combination of” or “administered along with” may be used interchangeably and mean that two or more agents are administered in the course of therapy. The agents may be administered together at the same time or separately in spaced apart intervals. The agents may be administered in a single dosage form or in separate dosage forms.

“Patient in need thereof” may include individuals, e.g., mammals such as humans, or canines, felines, porcines, rodents, etc., that have been diagnosed with a seizure disorder such as epilepsy, epilepsy with generalized tonic-clonic seizures, epilepsy with myoclonic absences, focal epilepsy, intractable focal epilepsy, focal cortical dysplasia, frontal lobe epilepsy, temporal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, brain tumor induced seizures, hamartoma induced seizures, drug withdrawal induced seizures, alcohol withdrawal induced seizures, increased seizure activity or breakthrough seizures (also called serial or cluster seizures). Seizure disorders can be associated with a sodium channel protein type 1 subunit alpha (Scn1a)-related disorder. The methods may be provided to any individual including, e.g., wherein the patient is a neonate, infant, a pediatric patient (6 months to 12 years), an adolescent patient (age 12-18 years) or an adult (over 18 years).

“Prodrug” refers to a pharmacological substance (drug) that is administered to a patient in an inactive (or significantly less active) form. Once administered, the prodrug is metabolized in the body (in vivo) into a compound having the desired pharmacological activity.

“Analog” and “Derivative” may be used interchangeably and refer to a compound that possesses the same core as the parent compound, but may differ from the parent compound in bond order, the absence or presence of one or more atoms and/or groups of atoms, and combinations thereof. Enantiomers are examples of derivatives. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

The term “pharmaceutically acceptable salt”, as used herein, refers to derivatives of the compounds defined herein, wherein the parent compound is modified by making acid or base salts thereof. Examples of pharmaceutically acceptable salts include, but are not limited to, nontoxic base addition salts with inorganic bases. Suitable inorganic bases such as alkali and alkaline earth metal bases include metallic cations such as sodium, potassium, magnesium, calcium and the like. The pharmaceutically acceptable salts can be synthesized from the parent compound by conventional chemical methods.

### EXAMPLES

The examples provided herein are included solely for augmenting the disclosure herein and should not be considered to be limiting in any respect.

### Example I

### Prospective Assessment of Efficacy of Chemogenetic Treatment in Mouse Seizure Model

On day one, 50 mice will be injected with kainic acid (KA, 200 ng/nl) in the hippocampus under isoflurane anesthesia. On day 21, AAV2/7-pAM/CaMKII-hM4D(Gi)-WPRE-BGHpA, (4.66 E+13 genome copies/ml) will be injected in the sclerotic hippocampus (500 nl). In 25 IHKA control mice AAV2/7-pAM/CaMKII-pL-WPRE-bGHpA control vector will be injected. At the same time a bipolar recording electrode will be implanted in the hippocampus. On day 42, mice with frequent hippocampal paroxysmal discharges (15 to 20 seizures/hour) will be selected for evaluation of perlapine effects. About 20% to 25% of the animals (5 to 6 mice/group) will be examined. EEG will continuously be recorded and recorded signals will be further processed to quantify the number of seizures. Perlapine will be administered at 0.1 mg/kg, 0.5 mg/kg, 1 mg/kg, 3 mg/kg, 5 mg/kg, 7 mg/kg and 10 mg/kg doses. Injections will be done intraperitoneally with at least 3 full days between individual doses (approximate duration 10 days). Mice will be evaluated to determine which dosages provide prolonged seizure suppression without side effects. An endpoint is at least 8 hours seizure suppression. Once optimal dose is determined chronic administration will be evaluated aiming at 1-3 doses per day for at least 5 days.

### Example 2

### Prospective Assessment of Efficacy of Ultrasound Enhanced Chemogenetic Treatment in Mouse Seizure Model

On day one, 50 mice will be injected with kainic acid (KA, 200 ng/nl) in the hippocampus under 2% isoflurane anesthesia. On day 21, AAV2/7-pAM/CaMKII-hM4D(Gi)-WPRE-BGHpA (4.66 E+13 genome copies/ml) will be injected in the sclerotic hippocampus (500 nl). In 25 IHKA control mice AAV2/7-pAM/CaMKII-pL-WPRE-bGHpA control vector will be injected. On day 42, mice with frequent hippocampal paroxysmal discharges (15 to 20 seizures/hour) will be selected for evaluation of perlapine and ultrasound effects. About 20% to 25% of the animals (5 to 6 mice/group) will be examined. EEG will continuously be recorded and recorded signals will be further processed to quantify the number of seizures.

Prior to perlapine administration, ultrasound energy will be applied to the BBB proximate to the hippocampal locus of the modified receptors. Each mouse will be anesthetized using 2% isoflurane and placed prone with its head immobilized by a stereotaxic apparatus that includes a mouse head holder, ear bars, and a gas anesthesia mask. The mouse hair will be removed using an electric trimmer and a depilatory cream. A degassed water-filled container sealed at the bottom with a thin, acoustically and optically transparent plastic wrap will be placed on top of the mouse head. Ultrasound coupling gel will also be used to eliminate any remaining impedance mismatch.

Ultrasound waves will be generated by a single-element spherical segment focused ultrasound transducer (center frequency: 1.525 MHz, focal depth: 90 mm, radius: 30 mm, available, e.g., from Riverside Research Institute, New York, N.Y., USA). A pulse-echo diagnostic transducer (center frequency: 7.5 MHz, focal length: 60 mm) will be aligned through a central, circular hole (radius 11.2 mm) of the focused ultrasound transducer so that the foci of the two transducers fully overlap. A cone filled with degassed and distilled water will be mounted onto the transducer system with the water contained in the cone by an acoustically transparent polyurethane membrane cap. The transducer system will be attached to a computer-controlled, three-dimensional positioning system (e.g., available from Velmex Inc., Lachine, QC, CAN). The focused ultrasound transducer will be connected to a matching circuit and driven by a computer-controlled function generator and a 50-dB power amplifier. The pulse-echo transducer will be driven by a pulser-receiver system connected to a digitizer in a personal computer.

The focused ultrasound transducer will be submerged in the degassed water-filled container with its beam axis perpendicular to the surface of the skull. The focus of the transducer will be positioned inside the mouse brain using, e.g., a grid-positioning method. The beam axis of the transducer will be aligned such that the focal point is placed 3 mm beneath the top of the parietal bone of the skull. In this placement, the focus of the focused ultrasound beam will overlap with the left hippocampus and the left posterior cerebral artery (PCA). The right hippocampus will not be targeted and can be used as a control.

A 25 μl bolus of ultrasound contrast agents constituting of microbubbles (mean diameter: 3.0-4.5 μm, concentration: 5.0-8.0×10⁸ bubbles per ml) will be injected into the tail vein 1-4 minutes prior to sonication. Pulsed focused ultrasound (pulse rate: 10 Hz, pulse duration: 20 ms, duty cycle: 20%) will then be applied at 0.64 MPa peak-to-peak in a series of two bursts consisting of 30 s of sonication at a single location (i.e., the hippocampus). Between each burst, a 30-s interval will be allowed for any residual heat between pulses to dissipate. The focused ultrasound sonication procedure can be performed one or more times in each mouse brain.

Following BBB opening, a bipolar recording electrode will be implanted in the hippocampus. Perlapine will then be administered at 0.1 mg/kg, 0.5 mg/kg, 1 mg/kg, 3 mg/kg, 5 mg/kg, 7 mg/kg and 10 mg/kg doses. Injections will be administered intraperitoneally with at least 3 full days between individual doses (approximate duration 10 days). Mice will be evaluated to determine which dosages provide prolonged seizure suppression without side effects. An endpoint is at least 8 hours seizure suppression. Once optimal dose is determined chronic administration will be evaluated aiming at 1-3 doses per day for at least 5 days.

### Example 3

### Prospective Assessment of Efficacy of Chemogenetic Treatment in Mouse Seizure Model

On day one, 50 mice will be injected with kainic acid (KA, 200 ng/nl) in the hippocampus under isoflurane anesthesia. On day 21, AAVRec3-pAM/CaMKII-hM4D(Gi)-WPRE-BGHpA, (4.66 E+13 genome copies/ml) will be injected in the sclerotic hippocampus (500 nl). In 25 IHKA control mice AAVRec3-pAM/CaMKII-pL-WPRE-bGHpA control vector will be injected. At the same time a bipolar recording electrode will be implanted in the hippocampus. On day 42, mice with frequent hippocampal paroxysmal discharges (15 to 20 seizures/hour) will be selected for evaluation of CNO effects. About 20% to 25% of the animals (5 to 6 mice/group) will be examined. EEG will continuously be recorded and recorded signals will be further processed to quantify the number of seizures. CNO will be administered at 0.1 mg/kg, 0.5 mg/kg, 1 mg/kg, 3 mg/kg, 5 mg/kg, 10 mg/kg and 15 mg/kg doses. Injections will be done intraperitoneally with at least 3 full days between individual doses (approximate duration 10 days). Mice will be evaluated to determine which dosages provide prolonged seizure suppression without side effects. An endpoint is at least 8 hours seizure suppression. Once optimal dose is determined chronic administration will be evaluated aiming at 1-3 doses per day for at least 5 days.

### Example 4

### Prospective Assessment of Efficacy of Ultrasound Enhanced Chemogenetic Treatment in Mouse Seizure Model

On day one, 50 mice will be injected with kainic acid (KA, 200 ng/nl) in the hippocampus under 2% isoflurane anesthesia. On day 21, AAVRec3-pAM/CaMKII-hM4D(Gi)-WPRE-BGHpA (4.66 E+13 genome copies/ml) will be injected in the sclerotic hippocampus (500 nl). In 25 IHKA control mice AAVRec3-pAM/CaMKII-pL-WPRE-bGHpA control vector will be injected. On day 42, mice with frequent hippocampal paroxysmal discharges (15 to 20 seizures/hour) will be selected for evaluation of CNO and ultrasound effects. About 20% to 25% of the animals (5 to 6 mice/group) will be examined. EEG will continuously be recorded and recorded signals will be further processed to quantify the number of seizures.

Prior to CNO administration, ultrasound energy will be applied to the BBB proximate to the hippocampal locus of the modified receptors. Each mouse will be anesthetized using 2% isoflurane and placed prone with its head immobilized by a stereotaxic apparatus that includes a mouse head holder, ear bars, and a gas anesthesia mask. The mouse hair will be removed using an electric trimmer and a depilatory cream. A degassed water-filled container sealed at the bottom with a thin, acoustically and optically transparent plastic wrap will be placed on top of the mouse head. Ultrasound coupling gel will also be used to eliminate any remaining impedance mismatch.

Ultrasound waves will be generated by a single-element spherical segment focused ultrasound transducer (center frequency: 1.525 MHz, focal depth: 90 mm, radius: 30 mm, available, e.g., from Riverside Research Institute, New York, N.Y., USA). A pulse-echo diagnostic transducer (center frequency: 7.5 MHz, focal length: 60 mm) will be aligned through a central, circular hole (radius 11.2 mm) of the focused ultrasound transducer so that the foci of the two transducers fully overlap. A cone filled with degassed and distilled water will be mounted onto the transducer system with the water contained in the cone by an acoustically transparent polyurethane membrane cap. The transducer system will be attached to a computer-controlled, three-dimensional positioning system (e.g., available from Velmex Inc., Lachine, QC, CAN). The focused ultrasound transducer will be connected to a matching circuit and driven by a computer-controlled function generator and a 50-dB power amplifier. The pulse-echo transducer will be driven by a pulser-receiver system connected to a digitizer in a personal computer.

The focused ultrasound transducer will be submerged in the degassed water-filled container with its beam axis perpendicular to the surface of the skull. The focus of the transducer will be positioned inside the mouse brain using, e.g., a grid-positioning method. The beam axis of the transducer will be aligned such that the focal point is placed 3 mm beneath the top of the parietal bone of the skull. In this placement, the focus of the focused ultrasound beam will overlap with the left hippocampus and the left posterior cerebral artery (PCA). The right hippocampus will not be targeted and can be used as a control.

A 25 μl bolus of ultrasound contrast agents constituting of microbubbles (mean diameter: 3.0-4.5 μm, concentration: 5.0-8.0×10⁸ bubbles per ml) will be injected into the tail vein 1-4 minutes prior to sonication. Pulsed focused ultrasound (pulse rate: 10 Hz, pulse duration: 20 ms, duty cycle: 20%) will then be applied at 0.64 MPa peak-to-peak in a series of two bursts consisting of 30 s of sonication at a single location (i.e., the hippocampus). Between each burst, a 30-s interval will be allowed for any residual heat between pulses to dissipate. The focused ultrasound sonication procedure can be performed one or more times in each mouse brain.

Following BBB opening, a bipolar recording electrode will be implanted in the hippocampus. CNO and an MRI contrast agent, e.g., gadolinium, will be administered simultaneously. CNO will be administered at 0.1 mg/kg, 0.5 mg/kg, 1 mg/kg, 3 mg/kg, 5 mg/kg, 7 mg/kg and 10 mg/kg doses. Injections will be administered intraperitoneally with at least 3 full days between individual doses (approximate duration 10 days).

The contrast agent will be used to determine whether the BBB has been opened by the focused ultrasound treatment. The agent will be observed by use of TI- and T2-weighted MRI scans using a 9.4 T system. The mice will be placed in a plastic tube with a 3.8-cm diameter birdcage coil attached and were inserted vertically into the magnet. Approximately 15 minutes after sonication, but before MRI contrast agent injection, a TI-weighted spin-echo MRI scan will be obtained (TR/TE: 246.1 ms/10 ms; BW: 50,505.1 Hz; matrix size: 256.times.256; FOV: 1.92.times.1.92 cm; slice thickness: 0.6 mm: NEX: 10, 15 and 45). Once the first scan is completed, 0.5 mL of MRI contrast agent gadolinium is administered intraperitoneally via a catheter to depict BBB opening. Intraperitoneal injection allows for the slow uptake of the MRI contrast agent into the bloodstream. After injection of the MRI contrast agent, a series of six alternating T1-weighted and T2-weighted fast spin-echo image scans (TR/TE: 4000 ms/9.2 ms; rapid acquisition with relaxation enhancement: 16; FOV: 1.92.times.1.92 cm; matrix size: 256.times.256; number of slices: 10; slice thickness: 0.6 mm; slice gap: 0.1 mm; NEX: 10, 15 and 45) are taken for each mouse.

Mice will be evaluated to determine which dosages provide prolonged seizure suppression without side effects. An endpoint is at least 8 hours seizure suppression. Once optimal dose is determined chronic administration will be evaluated aiming at 1-3 doses per day for at least 5 days.

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

What is claimed is:

1. A method of treating a seizure disorder in a patient in need thereof comprising:
administering to the patient an adeno-associated virus vector encoding hM4Di for delivery of hM4Di to a target location, the vector including a murine CaMKII promoter or a human CaMK2A promoter, a woodchuck hepatitis virus post-transcriptional regulatory element, and a bovine growth hormone polyadenylation sequence; and
administering to the patient a synthetic ligand which activates hM4Di.

2. The method of treating a seizure disorder according to claim 1, wherein the vector includes two inverted terminal repeats, a SV40 origin of replication, a pUC19 origin of replication, and an ampicillin resistance gene.

3. The method of treating a seizure disorder according to claim 2, wherein the vector is pAM/CaMKII-hM4D(Gi)-WPRE-BGHpA.

4. The method of treating a seizure disorder according to claim 2, wherein the vector is pAM/hCaMK2A-hM4D(Gi)-WPRE-BGHpA.

5. The method of treating a seizure disorder according to claim 1, wherein the vector includes a fluorescence reporter gene.

6. The method of treating a seizure disorder according to claim 1, wherein the adeno-associated virus vector is AAV1, AAV2, AAV4, AAV5, AAV7, AAV8 or AAV9.

7. The method of treating a seizure disorder according to claim 1, wherein the adeno-associated virus vector is AAVRec3.

8. The method of treating a seizure disorder according to claim 1, wherein the synthetic ligand is clozapine N-oxide.

9. The method of treating a seizure disorder according to claim 1, wherein the synthetic ligand is perlapine.

10. The method of treating a seizure disorder according to claim 1, wherein the vector is delivered to a target location in the patient's brain.

11. The method of treating a seizure disorder according to claim 1, wherein the vector is administered via a route selected from the group consisting of oral, buccal, sublingual, rectal, topical, intranasal, vaginal and parenteral.

12. The method of treating a seizure disorder according to claim 9, wherein the vector is administered directly to the target location.

13. The method of treating a seizure disorder according to claim 1, wherein the synthetic ligand is administered via a route selected from the group consisting of oral, buccal, sublingual, rectal, topical, intranasal, vaginal and parenteral.

14. The method of treating a seizure disorder according to claim 1, wherein the seizure disorder is selected from the group consisting of focal cortical dysplasia, epilepsy, epilepsy with generalized tonic-clonic seizures, epilepsy with myoclonic absences, frontal lobe epilepsy, temporal lobe epilepsy, occipital lobe epilepsy, parietal lobe epilepsy, Landau-Kleffner Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, brain tumor induced seizures, hamartoma induced seizures, drug withdrawal induced seizures, alcohol withdrawal induced seizures, increased seizure activity and breakthrough seizures.

15. The method of treating a seizure disorder according to claim 1, wherein the seizure disorder is characterized by focal seizures.

16. The method of treating a seizure disorder according to claim 1, wherein the method provides improvement in at least one symptom selected from the group consisting of ataxia, gait impairment, speech impairment, vocalization, involuntary laughter, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures

17. The method of treating a seizure disorder according to claim 1, further comprising applying ultrasound to a target location in the patient's brain to enhance permeability of the patient's blood brain barrier at the target location, wherein the vector is delivered to the target location.

18. The method of treating a seizure disorder according to claim 17, wherein the ultrasound is administered through the skull.

19. The method of treating a seizure disorder according to claim 17, further comprising exposing dura matter of the brain and delivering the ultrasound at or below the exposed dura matter.

20. The method of treating a seizure disorder according to claim 17, wherein the ultrasound is administered to the target location in the patient's brain prior to administering the vector.

21. The method of treating a seizure disorder according to claim 17, wherein the vector is administered prior to applying the ultrasound.

22. The method of treating a seizure disorder according to claim 17, wherein the ultrasound is administered to the target location in the patient's brain prior to administering the synthetic ligand.

23. The method of treating a seizure disorder according to claim 17, further comprising introducing a contrast agent into the patient, allowing sufficient time for the contrast agent to permeate the blood brain barrier and determining whether the contrast agent is present in the target location

24. A vector comprising nucleic acid encoding hM4Di under regulatory control of a murine CaMKII promoter or a human CaMK2A promoter, a woodchuck post-transcriptional regulatory element and a bovine growth hormone polyadenylation sequence.

25. The vector according to claim 24, further comprising two inverted terminal repeats, a SV40 origin of replication, a pUC19 origin of replication, and an ampicillin resistance gene.

26. The vector according to claim 24, wherein the vector is pAM/CaMKII-hM4D(Gi)-WPRE-BGHpA.

27. The vector according to claim 24, wherein the vector is pAM/hCaMK2A-hM4D(Gi)-WPRE-BGHpA.


SEQUENCE LISTING (5 sequences)

SEQ ID NO: 1 - DNA, 7169 bp, Artificial Sequence
  Synthetic plasmid construct, pAM/CaMKII- h4MD(Gi)-WPRE-BGHpA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtactcgagcattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgcttcagcatcccagccctagttcccagggatccaagctgaattcatggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactgcgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctggcttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctggccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggtagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 2 - protein, 738 aa, Adeno-associated virus
  misc_feature: AAVRec3 Amino Acid Sequence
MAADGYLPDWLEGNLSEGIREWWDLKPGAPKPKANQQKQDDGRGLVLPGYRYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 3 - DNA, 7209 bp, Artificial Sequence
  Synthetic plasmid construct, pAM/hCaMK2A- h4MD(Gi)-WPRE-BGHpA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtactcgaggtcgacggtatcgcgataagcttgatatcgaattccacggggttaatcgaataaataaataaataaataatataaataataaatgtccaggaatcagagctcaaactcagatccttagtcttaaactccagtcccttttcttcctaactccaagaccttggagtaagatcttgtggctgtaggtatggctgatgccctgaagagttgaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagaccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtggtccctaggttcgggagcagagcagcagcgcccccgggggatccaagctgaattcatggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactgcgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctggcttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctggccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggtagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 4 - DNA, 33 bp, Artificial Sequence
  Synthetic forward primer
atctaggaattcatggccaacttcacacctgtc

SEQ ID NO: 5 - DNA, 36 bp, Artificial Sequence
  Synthetic Reverse primer
atctagaagcttctacctggcagtgccgatgttccg